(19) United States
(12) Patent Application Publication
(10) Pub. No.: US 2025/0014677 A1
(43) Pub. Date: Jan. 9, 2025

(54) JOINT PROFILING OF GENETIC VARIANTS, DNA METHYLATION, GPC METHYLTRANSFERASE FOOTPRINTS, 3D GENOME AND TRANSCRIPTOME

(71) Applicant: Children's Hospital Medical Center, Cincinnati, OH (US)

(72) Inventors: LI WANG, Cincinnati, OH (US); YAPING LIU, Cincinnati, OH (US)

(73) Assignee: Children's Hospital Medical Center, Cincinnati, OH (US)

(21) Appl. No.: 18/712,405

(22) PCT Filed: Nov. 22, 2022
(86) PCT No.: PCT/US2022/050691

§ 371 Date: May 22, 2024

Related U.S. Application Data
(60) Provisional application No. 63/282,883, filed on Nov. 24, 2021.

(51) Int. Cl. G16B 20/20; C12Q 1/48; C12Q 1/6806; C12Q 1/6827; C12Q 1/6869

(52) U.S. Cl. CPC G16B 20/20; C12Q 1/48; C12Q 1/6806; C12Q 1/6827; C12Q 1/6869; C12Q 2600/154; C12Q 2600/156; G01N 2333/91057

(57) ABSTRACT

The present invention provides a system and methods for detecting long-range cis-regulatory element (CRE) activities in a nucleic acid molecule obtained from a cell sample, the system comprising: one or more components for measuring multi-omics from a single nucleic acid molecule obtained from the cell sample, wherein the multi-omics measurements comprise one or more of: three-dimensional chromosomal conformation, CpG methylation, GpC accessibility, single nucleotide polymorphisms (SNPs); or one or more combinations thereof, and one or more components for measuring a transcriptome of the cell sample; wherein the system is capable of profiling a plurality of long-range CREs within the nucleic acid molecule obtained from the cell sample.

### CROSS REFERENCE TO RELATED APPLICATIONS

This application claims the benefit of U.S. Provisional Application No. 63/282,883, filed Nov. 24, 2021, the entirety of which is incorporated by reference herein.

### GOVERNMENT RIGHTS

This invention was made with government support under grant number R35GM147283 awarded by the National Institutes of Health. The government has certain rights in the invention.

### TECHNICAL FIELD

The present disclosure relates to methods for simultaneously capturing the single nucleotide polymorphisms, DNA methylation, GpC methyltransferase footprints, and chromosome conformation changes from a single DNA molecule, together with transcriptome in the same assay. The methods or workflow of the present disclosure are referred to as GTAGMe-seq. The single DNA molecule can be isolated and purified from a bulk cell sample or from a single cell sample.

### BACKGROUND

Cis-regulatory elements (CREs) play instrumental roles in the regulation of mammalian gene expression. Sets of transcriptional factors (TFs) bring together multiple CREs, which can be up to megabases away, to initiate and maintain the expression of their targeted genes. GpC methyltransferase is recently utilized to footprint the TF bindings at CREs and reveals the coordinated activities of CREs locally. Since DNA is spatially organized into three-dimensional (3D) space in nuclei, distal CREs can be brought into proximity through chromatin folding. Therefore, it is possible that spatially proximal regions may also exhibit a coordinated GpC methyltransferase footprint. However, conventional GpC methyltransferase-based approaches, such as NOMe-seq/dSMF, have limited power in detecting coordinated CREs activities across large distances due to the short reads profiled in next-generation sequencing (NGS). Recent SMAC-seq combined methyltransferase with Nanopore sequencing to detect the coordinated activities up to many kilobases, which, however, did not provide the spatial proximity information to distinguish the cis-co-binding events by the same set of TFs with that due to the trans-effects.

Moreover, recent studies suggested that allelic differences in genetic content can affect the local CREs' activity by using heterozygous SNPs and their linked CpG methylation or GpC methyltransferase footprint from the same reads. In principle, the genetic differences between two alleles may affect the coordination activities of both CREs that are distant in linear 2D consideration of chromosomes but proximate in spatially in 3D. However, the current understanding of the long-range allele-specific CRE activities is still limited by both computational approaches and experimental assays. First, most GpC methyltransferase-based assays heavily depend on the sodium bisulfite treatment, which will lead to difficulties in distinguishing C>T SNPs (65% SNPs in dbSNP) and C>T substitutions caused by bisulfite conversion. Previously developed Bis-SNP makes it possible to characterize the SNPs at limited known SNP loci with high accuracy in single bisulfite sequencing. However, the genome-wide genotyping accuracy is still not comparable to the whole genome sequencing (WGS). Second, both the short fragment sizes in NGS libraries and the lack of spatial proximity information in long-read sequencing will prevent one from characterizing the spatial interactions between genetic contents and distal CRE activities at two different alleles. Accordingly, there is a need for improved techniques for determining how multiple CREs coordinate together to regulate downstream gene expression. The present invention provides methods for measuring long-range allele-specific CRE activities.

### SUMMARY

In certain aspects, the present invention provides a system for detecting long-range cis-regulatory element (CRE) activities in a nucleic acid molecule obtained from a cell sample, the system comprising: one or more components for measuring multi-omics from a single nucleic acid molecule obtained from the cell sample, wherein the multi-omics measurements comprise one or more of: three-dimensional chromosomal conformation, CpG methylation, GpC accessibility, single nucleotide polymorphisms (SNPs); or one or more combinations thereof, and one or more components for measuring a transcriptome of the cell sample; wherein the system is capable of profiling a plurality of long-range CREs within the nucleic acid molecule obtained from the cell sample.

In some embodiments, the cell sample comprises a bulk cell sample.

In some embodiments, the cell sample comprises a single cell sample.

In some embodiments, the one or more components for measuring three-dimensional chromosome conformation perform in situ methyl-HiC.

In some embodiments, the one or more components for measuring CpG methylation perform one or more of bisulfite conversion and paired-end sequencing.

In some embodiments, the one or more components for measuring the GpC accessibility comprises perform GpC methyltransferase foot-printing.

In some embodiments, the GpC methyltransferase foot-printing is performed using one or more GpC methyltransferases comprising M CviPI.

In some embodiments, the one or more components for measuring SNPs perform Bis-SNP analysis, a PairHMM based analysis, or a combination thereof.

In some embodiments, the one or more components for measuring transcriptome perform an allele-specific transcriptome comparison of the two alleles of a single chromosome.

In certain aspects, the present invention provides a method for detecting long-range CREs, the method comprising: obtaining a cell sample; performing nucleic acid cross-linking using one or more techniques; isolating a nuclear sample from the cell sample; measuring the methyltransferase footprint of the nuclear sample, separating the nuclear samples into a first aliquot and a second aliquot, preparing one or more RNA libraries from the nuclear sample in the first aliquot; and, preparing one or more DNA libraries from the nuclear sample in the second aliquot.

In some embodiment, the cell sample comprises a single cell sample.

In some embodiment, the cell sample comprises a bulk cell sample. In some embodiment, the DNA library is a bisulfite converted DNA library. In some embodiment, the method further comprises paired-end sequencing the bisulfite converted DNA library.

In some embodiments, the method further comprises quantifying the transcript abundance in the first aliquot. In some embodiments, the transcript abundance is determined using one or more of alignment-based techniques, alignment-free techniques, or both alignment-based and alignment free techniques.

In some embodiments, the RNA library is prepared using one or more Tn5-transposase-based techniques.

In some embodiments, the RNA library is prepared using one or more one or more combinations of enzymatic fragmentation and adaptor addition.

In some embodiment, the one or more DNA libraries are deeper sequences using one or more techniques comprising 150 bp paired-end sequencing.

In certain aspects, the present invention provides a computation method for analyzing a single-molecule nucleic acid sample comprising processing a cell sample using the system of claim 1 for characterizing or quantifying one or more of DNA methylation, GCH methyltransferase accessibility, three-dimensional genomic conformation, transcriptome, and one or more combinations thereof.

### BRIEF DESCRIPTION OF THE DRAWINGS

FIGS. 1A-1G depict an exemplary workflow as contemplated by the present invention, demonstrating GTAGMe-seq generating high-quality multi-omics data. FIG. 1A depicts a schematic of an exemplary method as contemplated herein. FIG. 1B depicts the 3D genome concordance with Hi-C in IMR-90. From left to right: 1 Mb resolution across all chromosomes, 250 kb resolution for chromosome 6 (include a comparison of compartment score with Hi-C), 50 kb resolution for chromosome 6 (include a comparison of insulation score with Hi-C). FIG. 1C depicts the methylation concordance with WGBS at HCG (H=A, C, or T) sites in IMR-90. FIG. 1D depicts the average GCH (H=A, C, or T) methyltransferase footprint level (left) and HCG methylation level (right) near PolII binding sites. PolII binding sites are divided into quantiles based on the signal strengths of PolII ChIP-seq in IMR-90. Matched random intervals are randomly chosen from the same chromosome and interval lengths as the intervals in top 0-25% quantile. FIG. 1E depicts the average GCH methyltransferase footprint level and HCG methylation level around the distal CEBPB binding sites in IMR-90. FIG. 1F depicts an exemplary scatterplot of the concordance at the gene level with total RNA-seq (ENCODE) in IMR-90. FIG. 1G depicts the SNPs concordance with the ground truth SNPs data from Genome in a Bottle (GIAB). The results are compared with the SNPs called from WGS (1000 genome project, phase 3 in NA12878). Ti/Tv ratio is also compared.

FIGS. 2A and 2B depict array data showing that GTAGMe-seq reveals the coordinated GCH methyltransferase footprint at distal regions in spatial proximity. FIG. 2A demonstrates that the coordinated GCH methyltransferase footprint is much stronger at the read pairs from the same DNA molecules (top panel) than that from different DNA molecules but in the same regions (bottom panel). Each dot at the heatmap represents one read pair within the HiCCUPS anchor regions. FIG. 2B depicts the average GCH methyltransferase concordance level across all the HiCCUPS anchor regions (>20 kb, orange color), their matched local regions (utilize the read pairs from the same anchor and within 1 kb distance, black color), or matched shuffled read pairs from the same HiCCUPS anchor regions (>20 kb, blue color).

FIGS. 3A and 3B depicts long-range allele-specific GCH methyltransferase footprint data obtained using GTAGMe-seq. FIG. 3A depicts a scheme and the heatmap representations of the three groups' long-range GCH methyltransferase footprint. The distance between SNP anchors and non-SNP anchors should be more than 1 kb. Group 1 (top) is the group of loci with significant differences of GCH methyltransferase footprint between Allele A and Allele B at both SNP anchors (FDR<0.05) and non-SNP anchors (FDR<0.05). Group 2 (middle) is the group of loci with significant differences of GCH methyltransferase footprint between Allele A and Allele B only at SNP anchors (FDR<0.05) but not at non-SNP anchors (FDR>0.95). Group 3 (bottom) is the group of loci with significant differences of GCH methyltransferase footprint between Allele A and Allele B only at non-SNP anchors (FDR<0.05) but not at SNP anchors (FDR>0.95). The rows of the heatmap are clustered by columns marked with the magenta star (*). FIG. 3B depict the log 2 enrichment of TF binding sites at SNP anchors and non-SNP anchors. The log 2 ratio enrichment level is calculated by the ratio with the matched random intervals in the same chromosomes. The random permutation is repeated ten times. Only the top 3 enriched TF binding sites are shown.

FIGS. 4A-4K depict multi-omics data generated from GTAGMe-seq from different cell types. FIG. 4A depicts a comparison of contact frequency distance decay curves obtained from in situ Hi-C and GTAGMe-seq data at IMR-90 and GM12878. FIG. 4B depicts a comparison of compartment scores obtained from in situ Hi-C and GTAGMe-seq data at IMR-90 (left) and GM12878 (right). FIG. 4C depicts a comparison of insulation scores obtained from in situ Hi-C and GTAGMe-seq data at IMR-90 (left) and GM12878 (right). FIG. 4D depicts matrix similarity between in situ Hi-C and GTAGMe-seq measured by stratum adjusted correlation coefficient (SCC) from HiCRep at different resolutions in IMR-90 (left) and GM12878 (right). FIG. 4E depicts the overlap of TADs obtained from in situ Hi-C and GTAGMe-seq data at IMR-90 (left) and GM12878 (right). FIG. 4F depicts the methylation concordance with WGBS at HCG (H=A, C, or T) sites in GM12878. FIG. 4G depicts the number of HCG sites covered by in situ Hi-C and GTAGMe-seq data at IMR-90 (left) and GM12878 (right). FIG. 4H depicts a scatterplot of the concordance at the gene level with total RNA-seq (ENCODE) in GM12878. FIG. 4I depicts a scatterplot of the concordance at the gene level between biological replicates in GTAGMe-seq data at IMR-90 (left) and GM12878 (right). FIG. 4J depicts the Ti/Tv ratio at gold standard data (GIAB) before or after imputation and at Bis-SNP results (filtered or raw) before and after imputation from GTAGMe-seq in NA12878. FIG. 4K depicts the percentage of concordant SNPs with the ground truth (GIAB) at the callable sites (with reads covered and genotypes called) in WGS and GTAGMe-seq (by Bis-SNP, filtered or raw) in NA12878 before or after the imputation.

FIGS. 5A-5E depict the average GCH methyltransferase footprint and HCG methylation level around distal binding sites for specific transcription factors in IMR-90. FIG. 5A shows results for CTCF. FIG. 5B shows results for FOS. FIG. 5C shows results for MAFK. FIG. 5D shows results for. MAZ. FIG. 5E shows results for MIX1. The TF binding sites are characterized by TF ChIP-seq in IMR-90.

FIGS. 6A and 6B depict data demonstrating that GTAGMe-seq reveals similar sets of differentially expressed genes as total RNA-seq. FIG. 6A depicts the overlap of significantly upregulated genes between IMR-90 and GM12878 that are characterized by total RNA-seq from two different labs in ENCODE (left), characterized by GTAGMe-seq and total RNA-seq from ENCODE (middle), or characterized by three datasets (right). FIG. 6B depicts the overlap of significantly downregulated genes between IMR-90 and GM12878 that are characterized by total RNA-seq from two different labs in ENCODE (left), characterized by GTAGMe-seq and total RNA-seq from ENCODE (middle), or characterized by three datasets (right).

FIG. 7 depicts GTAGMe-seq reveals the long-192 range allele-specific GCH methyltransferase footprint. The rows of the heatmap are clustered by all four columns (marked with a star *) as compared to being clustered in one column as shown in FIG. 3A.

FIGS. 8A-8G depicts exemplary results from single-nuclei GTAGMe-seq used to jointly profile multiomics within the same nuclei as contemplated by the present invention.

FIG. 8A depicts the concordance of DNA methylation between bulk GTAGMe-seq and pooled snGTAGMe-seq at each single HCG site(H=A, C, or T) in IMR-90 cell line. FIG. 8B depicts the comparison of pearson correlation map in 2.5 Mb resolution at chr15 between pooled snGTAGMe-seq and bulk GTAGMe-seq from an IMR-90 cell line. FIG. 8C depicts the GCH (H=A, C, or T) accessibility level from pooled snGTAGMe-seq (lower line) and bulk (upper line) GTAGMe-seq at Distal CEBPB binding sites (no TSS within 2 kb). FIG. 8D depicts the concordance of gene expression between bulk GTAGMe-seq and pooled snGTAGMe-seq. CPM: count per million. FIG. 8E depicts the distribution of DNA methylation level at HCGs from snGTAGMe-seq (positive control, 1000 cells, ˜0.8× with 2.3M fragments) and one of the snGTAGMe-seq (sc_18 library, ˜1.2× with 2.9M fragments). FIG. 8F depicts T-SNE by DNA methylation (Cluster 1 and Cluster 2), 3D genome (colored by DNA methylation cluster label), GCH accessibility near a single TFBS (FOS)(colored by DNA methylation cluster label) or gene expression (orange (upper left) and green (lower right)) can separate randomly mixed two cell types. Some cells were lost in the RNA-seq library preparation. FIG. 8G depicts the concordance of genotype between pooled snGTAGMe-seq clusters and bulk WGS suggests that snGTAGMe-seq can reveal the heterogeneity of genetic variants. Clusters are from t-SNE of DNA methylation in panel F. Genotyping result in bulk WGS is from public databases (GM12878, 1000G project) or GATK pipeline (IMR-90).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

The present invention provides systems and methods for simultaneously studying the genetic variants, DNA methylation, GpC methyltransferase footprint, and 3D genome in the same DNA molecules together with the transcriptome in the same assay. The present invention combines multiple assays allowing one for the first time to uncover the coordinated GpC methyltransferase footprint and long-range allele-specific footprint at genomic regions that are far away in linear DNA sequences but spatially close in the nucleus.

In some embodiments, the methods of the present invention provide a high-throughput assay that jointly profile genetic variants, DNA methylation, DNA accessibility, and 3D genome at the same DNA molecule, together with transcriptome in a single experiment. The method of the present invention as referred to herein is called GTAGMe-seq (Genetics, Transcriptome, Accessibility, 3D Genome, and Methylation sequencing).

In some embodiments the methods are applied to one or more samples of cells. The cells may include any suitable mammalian cell type as understood in the art. For example, in some embodiments the cells may include fibroblasts, lymphocytes, stem cells, monocytes, epithelial cells, endothelial cells, adipocytes, chondrocytes, osteocytes, granulocytes, neurons, and the like. In some embodiments the one or more samples of cells including one or more cell lines including GM12878, IMR-90, and mESC. In some embodiments, the methods are applied to one or more tissues isolated from one or more mammalian species including for example murine, rattus, canine, feline, bovine, equine, non-human primates, and the like. The one or more tissues may include cardiac tissue, prefrontal cortex tissue, muscle tissue, bone tissue, connective tissue, adipose tissue, or other suitable tissues as understood in the art.

In some embodiments of the methods include one or more steps for preserving the integrity of RNA molecules in the sample. In some embodiments, the integrity of the RNA is preserved by adding one or more RNase inhibitors to the sample. In some embodiments, the RNase inhibitors are added before any steps of the methods as contemplated herein are commenced. In some embodiments, the RNase inhibitors are added in between any two steps as contemplated herein. In some embodiments, one or more RNase inhibitors are added in one or more steps of the methods as contemplated herein. The RNase inhibitors may be added at least once, at least twice, at least three times, at least four times, at least five times, at least six times, and so on.

Embodiments of the methods include crosslinking the cells. In some embodiments, the cells are crosslinked with a crosslinking agent. In some embodiments, the crosslinking agent includes formaldehyde, acetone, and/or one or more other suitable agents.

In some embodiments the crosslinked cells are suspended in a nuclei isolation buffer. The nuclei isolation buffer may include one or more RNase inhibitors.

In some embodiments the methods include permeabilizing the nuclear membrane of the one or more cells. The nuclear membranes may be permeabilized with one or more suitable detergents as understood in the art.

Embodiments of the methods include digesting chromatin. In some embodiments, the chromatin is digested with one or more suitable restriction enzymes as understood in the art. In some embodiments the one or more restriction enzymes include methylation insensitive restriction enzymes, for example DpnII,

Embodiments of the methods include performing biotin fill-in. The biotin fill-in is performed using one or more suitable techniques as understood in the art. For example, in some embodiments the biotin fill-in is performed by inactivating the one or more restriction enzymes (e.g., DpnII). In some embodiments, the one or more restriction enzymes is inactivated by adding heat. In some embodiments the amount of heat includes up to 40° C., from about 40° C. to about 45° C., from about 45° to about 50° C., from about 50° C. to about 55° C., from about 55° C. to about 60° C., from about 60° C. to about 65° C., from about 65° C. to about 70°, from about 70° C. to about 75° C., from about 75° C. to about 80° C., including any and all increments therebetween. In some embodiments, the restriction enzymes is inactivated by adding heat to 65° C. In some embodiments, the sticky ends of the DNA are then filled in with biotin or one or more biotin formulations including formulations containing one or more nucleoside triphosphates (e.g., Biotin-1,4-dATP, dGTP, dCTP and dTTP) using one or more DNA polymerases (e.g., Klenow) under suitable conditions as understood in the art. For example, in some embodiments, the biotin fill-in is performed at 37° C. for a suitable duration of time. In some embodiments, the biotin fill-in is performed under agitation, rocking, stirring, mixing, shaking or the like. In some embodiment, the mixtures is supplemented with one or more RNase inhibitors and/or one or more recombinant ribonuclease inhibitors in order to prevent RNA degradation. In some embodiments, one or more reducing agents is also added (e.g. DTT).

Embodiments of the methods include performing in situ ligation of proximal segments using one or more suitable ligases including for example T4 DNA ligase. In some embodiments, the in situ ligation may be used to obtain 3D spatial proximal information.

In some embodiments, the nucleosomes and transcription factors are crosslinked with the DNA inside the nuclei. Accordingly, in some embodiments, the methods include footprinting the chromatin accessibility using one or more suitable enzymes including, for example GpC methyltransferase (M.CviPI).

Embodiments of the methods include separating the RNA using one or more suitable techniques to simultaneously extract both the DNA and RNA from the one or more crosslinked cells. In some embodiments, the one or more suitable techniques for separating DNA samples and RNA samples including MagMAX™ FFPE DNA/RNA Ultra Kit.

Embodiments of the methods include preparing a total RNA library from the separated or isolated RNA sample using one or more suitable tools as understood in the art. For example, in some embodiments the RNA library is prepared using SMARTer® Stranded Total RNA-Seq Kit v2. In some preferred embodiments, the RNA library is prepare using SMARTer® Stranded Total RNA-Seq Kit v2 as it utilizes template switching and extension strategies in order to at least partially recover some of the fragmentation of the RNA molecules.

Embodiments of the methods include reversing the crosslinking of the cells. In some embodiments, the crosslinking is reversed using one or more suitable means including for example, applying heat. In some embodiments the amount of heat includes up to 40° C., from about 40° C. to about 45° C., from about 45° to about 50° C., from about 50° C. to about 55° C., from about 55° C. to about 60° C., from about 60° C. to about 65° C., from about 65° C. to about 70°, from about 70° C. to about 75° C., from about 75° C. to about 80° C., including any and all increments therebetween. In some embodiments, the crosslinking is reversed by applying about 65° C.

Embodiments of the methods include precipitating the cellular DNA. In some embodiments, the DNA is precipitated using any suitable technique as understood in the art. For example, in some embodiments the DNA is precipitated using ethanol precipitation, isopropanol precipitation, and the like.

Embodiments of the methods include sonicating the precipitated DNA. The DNA may be sonicated in order to fragment the DNA into segments having a suitable length. In some embodiments, the DNA is sonicated so that the DNA is fragmented to the average length of about 400 bp. The DNA may be fragmented to an average length of up to about 200 bp, from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, and any an all increments or values therebetween.

Embodiments of the methods include cleaning up the fragmented DNA using one or more suitable techniques including for example bead purification. In some embodiments the DNA is cleaned up using, for example 1×AMPure beads.

Embodiments of the methods include pulling down biotin-marked DNA fragments using one or more suitable techniques including streptavidin beads. In some embodiments, the DNA fragments are pulled down in order to enrich proximal-ligated fragments, perform end-repair, d-A tailing, and ligate the DNA fragments with a truncated cytosine-methylated adapter.

Embodiments of the methods include analyzing the purified DNA samples using one or more suitable techniques. For example, embodiments of the methods include measuring the bisulfite conversion efficiency using one or more techniques including for example spiking into the sample unmethylated lambda DNA. The unmethylated lambda DNA may be “spiked in” at one or more concentrations including for example in the range of about 0.1%˜0.5%. In some embodiments, the unmethylated lambda DNA concentration is up to about 0.1%, from about 0.1% to about 0.2%, from about 0.2% to about 0.3% from about 0.3% to about 0.4%, from about 0.4% to about 0.5%, from about 0.5% to about 0.6%, from about 0.6% to about 0.7%, from about 0.7% to about 0.8%, from about 0.8% to about 0.9%, from about 0.9% to about 1.0%, and any and all increments therebetween. In some embodiments the unmethylated lambda DNA concentration is in the range of from about 0.1% about 0.5%, from about 0.05% to about 1%, from about 0.05% to about 0.75%, and any and all increments therebetween.

Embodiments of the methods including performing bisulfite conversion. The bisulfite conversion can be performed or conducted using the any suitable technique as understood in the art. For example, in some embodiments the bisulfite conversion is performed using a EZ DNA Methylation-Gold Kit. In some embodiments, the bisulfite converted DNA library is prepared using a KAPA HiFi Uracil+ ReadyMix kit.

Embodiments of the method include evaluating the quality of the DNA library using one or more suitable techniques. For example, the quality of the DNA library may be assessed using one or more of Qubit, qPCR, and BioAnalyzer 2000 (Agilent Technologies). In some embodiments, the quality of the DNA library is assessed by performing shallow coverage paired-end sequencing (<2 million reads) using, for example, Illumina MiSeq in order to check the library quality and complexity.

Some embodiments of the methods include more deeply performing paired-end sequencing using one or more techniques including for example, an Illumina HiSeq XTen or NovaSeq S4 6000 platform. In some embodiments, the RNA-seq libraries or Phi-X (20%) are spiked in to overcome the imbalance of GC ratio in DNA libraries caused by bisulfite treatment.

Embodiments of the methods of the present invention also provide a computational workflow for processing the GTAGMe-seq data. Embodiments of the computation workflow methods relate to processing data obtain from the DNA samples. The DNA data is processed by first performing one or more of FastQC and MultiQC at raw fastq files.

Embodiments of the methods include removing the adapters and low-quality reads using one or more techniques including Trim Galore!. In some embodiments, FastQC and MultiQC are again performed in order to check if the adapter contamination and low-quality reads have been removed.

Embodiments of the methods include preparing converted reference genomes. In some embodiments, two in silico converted reference genomes are prepared for the bisulfite reads mapping. In some embodiments the reference genomes are prepared by making a C to T reference (all C's were converted to T's) and a G to A reference (all G's were converted to A's). In some embodiments, the reads mapping pipeline is adapted from a previously developed bhmem which adapted and configured for Methyl-HiC. In some embodiments, only high-quality reads are kept for the later analysis. High quality reads are determined wherein both ends are uniquely mapped and mapQ>30 and wherein there are no PCR duplicated reads. In some embodiments, the reads with incompletely converted cytosine (e.g., WCH, W=A or T, H=A, C, or T) are filtered out.

Embodiments of the methods include performing a 3D genome analysis. In some embodiments, the 3D genome analysis includes converting Bam files to .hic files similar to Hi-C data. In some embodiments, the 3D genome analysis includes identifying DNA compartments. In some embodiments, the 3D genome analysis includes identifying topological associated domains (TADs). In some embodiments, the 3D genome analysis includes identifying chromatin loops. In some embodiments the one or more of DNA compartments, the topological associated domains (TADs), and the chromatin loops are identified and/or analyzed simultaneously. In some embodiments the one or more of DNA compartments, the topological associated domains (TADs), and the chromatin loops are identified and/or analyzed sequentially. In some embodiments one or more of DNA compartments, topological associated domains (TADs), and chromatin loops are identified and/or analyzed using one or more techniques or tools including, for example the Juicer 8 pipeline.

Embodiments of the methods include calculating DNA methylation and GpC methyltransferase accessibility. In some embodiments, DNA methylation and GpC methyltransferase accessibility are analyzed by calculating the HCG methylation and GCH accessibility level using one or more suitable techniques including for example Bis-SNP in NOMe-seq mode.

Embodiments of the methods relate to processing data obtain from the isolated RNA samples. Similar to the analysis performed on the isolated DNA samples, embodiments of the methods for processing the RNA samples include performing one more sample analysis techniques such as FastQC, MultiQC, and Trim Galore! for assessing the quality of the sample and/or for detecting potential problems with the RNA sample. In some embodiments, the isolated RNA is analyzed in order to remove adapters and low-quality reads. In some embodiments, the transcript abundance is quantified using one or more techniques. In some embodiments, the transcript abundance is quantified using one or more alignment-based techniques. The alignment-based techniques may include one or more of STAR and RNA-SeQC. In some embodiments, transcript abundance is quantified using one or more and alignment-free techniques. The alignment-free techniques may include, for example Salmon. In some embodiments, the transcript abundance is quantified using either alignment-based or alignment free techniques using reference transcriptome annotation from Gencode. In some embodiments, the isolated RNA is analyzed for G-C content. In some embodiments, one or more techniques are used to correct for G-C content and/or fragmentation bias. In some embodiments, the isolated RNA is analyzed in order to detect differential transcript expression between samples. In some embodiments, differential expression is analyzed using one or more suitable techniques at the gene level. For example, the differential expression can be measured using tools such as edgeR or similar tools.

Embodiments of the methods include identifying one or more genetic variants including single nucleotide polymorphisms (SNPs) and insertions and/or deletions (indels) using the GTAGMe-seq data. Embodiments of the methods include identifying the genetic variants with high accuracy.

Embodiments of the methods include identifying genetic variants in the DNA sample. In some embodiments, the genetic variants in the DNA sample are identified using a PairHMM-based technique, for example GATK4. In some embodiments, both SNPs and short insertions/deletions (indels) are identified in the bisulfite converted sample. In some embodiments, poorly identified SNPs and Indels with high strand bias and one or more other potential problems are filtered out. In some embodiments, using the filtered SNPs, a large reference panel is used to phase and impute the genotype. In some embodiments, only genotypes with high phasing and imputation scores are kept for allele-specific epigenetic analysis.

Embodiments of the methods include identifying genetic variants in the RNA sample. In some embodiments, the RNA sample is analyzed for genetic variants including, for example, the identification of SNPs and insertions and/or deletions (indels) in the RNA-seq data. In some embodiments, the RNA sample is analyzed for allele-specific genetic variant expression.

Embodiments of the methods include benchmarking or validating the performance of GTAGMe-seq data using mono-omic data. In some embodiments the mono-omic data is publicly available data. In some embodiments, the publicly available data is obtained using the same cell line, cell sample, or tissue sample as that being assayed using the GTAGMe-seq method as disclosed herein. In some embodiments, the publicly available data includes data obtained using cell lines, cell samples, or tissue samples including comprehensive profiles, genetic variants, epigenetic marks, and transcriptome. In some embodiments, the data is compared at one or more resolutions. For example, in some embodiments, the 3D genome results are analyzed at different resolution levels for evaluating different features including compartments (resolution: ˜500 kb-1 MB), TADs (resolution: ˜40 kb-200 kb), and chromatin loops (resolution: ˜5 kb-25 kb). In some embodiments, statistical analysis is further utilized. For example, in some embodiments, a stratum-adjusted correlation coefficient (SCC) is measured by HiCRep, to quantify the similarity between two datasets across different resolutions. In some embodiments, the gene expression is measured and/or validated by comparing the RNA abundance with public total RNA-seq at gene-level and transcript-level. In some embodiments, NOMe-seq and RNA-seq data is generated on the same cell line after crosslinking in order to verify the concordance of GpC methyltransferase accessibility and transcriptome. In some embodiments, the measurements of genetic variants are compared or validated. For example, in some embodiments, the SNPs and insertions/deletion (Indels) in DNA obtained from a specific cell line, cell sample, or tissue sample is compared with deep whole genome sequencing (WGS) from the 1000 genome project on the same sample. In some embodiments, the genetic variants are compared by ground truth genotype results from the same sample, similar sample, same type of sample, or similar type of sample (e.g., same or similar specific cell line, cell sample, or tissue sample). In some embodiments, the genotype concordance and transition (Ti) to transversion (Tv) (Tv/Ti) ratio are also assessed.

Embodiments of the methods include identify long-range (>20 kb) epigenetic concordance in GTAGMe-seq data. In some embodiments, in order to demonstrate the power of GTAMe-seq and understand the coordination of epigenetic status over a large genomic distance,

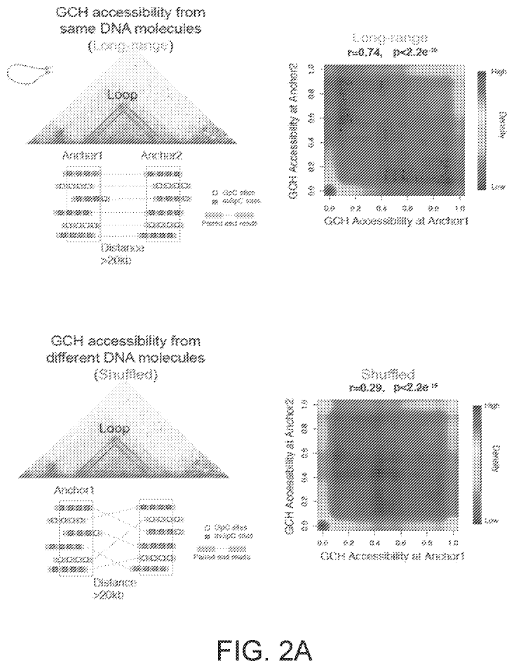

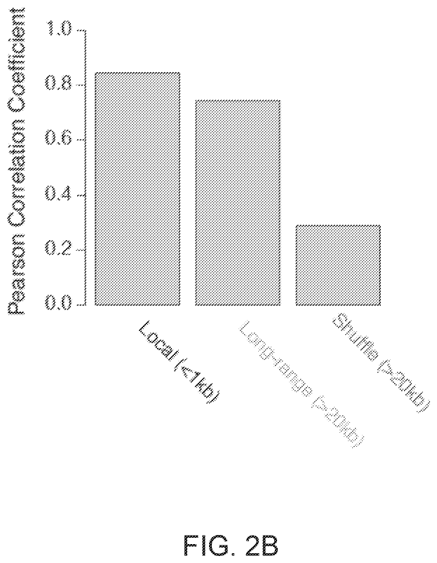

Embodiments of the methods include analyzing the DNA methylation and/or chromatin accessibility in the DNA sample. In some embodiments, one or more genomic regions linearly separated but positioned in proximity in the 3D genome topology are evaluated for coordinated DNA methylation and/or chromatin accessibility status. As shown in FIG. 2A, in some embodiments, the correlation coefficient of the GCH (H=A, C, or T) methyltransferase accessibility is calculated on GTAGMe-seq reads separated in chromatin loop anchors. For example, in order to check the coordination of chromatin accessibility, only read pairs with GCH (H=A, C, or T) methyltransferase accessibility levels at both ends are retained for analysis. For the regions of interest, such as chromatin loops' anchor, Pearson Correlation Coefficient (PCC) will be calculated directly from the GCH methylation level of each read of the read pair, but not by the average GCH methylation level at each end of anchor regions. Only read pairs spanning at least 20 kb genomic distance will be considered the long-range for the analysis.

In some embodiments, as a control, all the read pairs within the same genomic regions, no matter whether they have long-range interaction or not, are randomly shuffled one or more times and then subjected to PCC calculation (shown in FIG. 2B). In some embodiments one or statistical methods is applied, for example Fisher's (1925) z test. The one or more statistical methods are performed in order to assess the significance between observed PCC from interacted read pairs and PCC of the random shuffled read pairs.

Embodiments of the methods include assessing HCG methylation. In some embodiments, coordination analysis at the GCH accessibility level is performed between pairs of TFBS (˜200 bp to 1 kb resolution), as well as at one or more other genomic regions, including for example, enhancer-promoter links, imprinting regions, and CREs in sex chromosomes.

Embodiments of the methods include identifying long-range (>20 kb) allele-specific epigenetic events in GTAGMe-seq data.

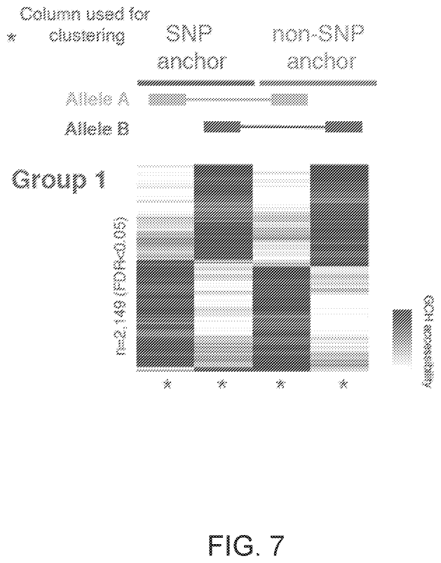

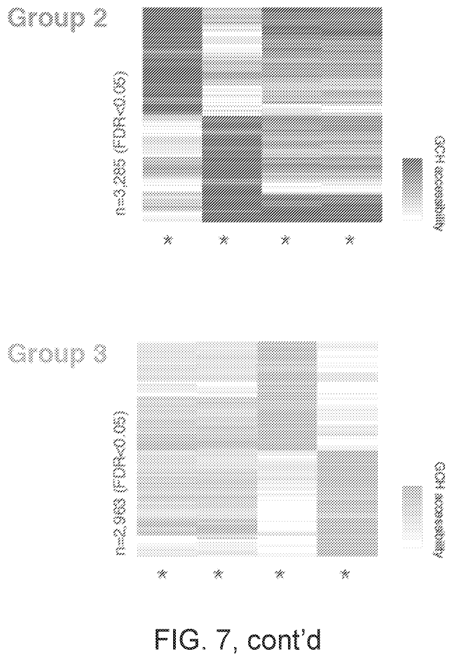

Embodiments of the methods include preforming allele-specific epigenetics analysis. In some embodiments, allele-specific epigenetic analysis, for example, chromatin accessibility, is performed locally near the genetic variants due to the limited lengths of short reads. In some embodiments, the long-range allele-specific HCG methylation, GCH methyltransferase accessibility, and transcriptome are analyzed in view of the spatial distance in the 3D genome topology. In some embodiments, the GTAGMe-seq reads are separated into two or more groups by their parent-of-origin at the heterozygous SNPs (SNP anchors, FIG. 7). In some embodiments, the DNA read pairs are separated into two alleles, even though they may not overlap with any SNPs mapped over large genomic distances (non-SNP anchors, FIG. 7).

Embodiments of the methods include validating the discovery of long-range epigenetic concordance and allele-specific epigenetic events. In some embodiments, a target-based GTAGMe-seq method is used to validate 10-20 genetic loci. In order to modify the GTAGMe-seq to a more targeted method, once the DNA is reverse crosslinked and separation, a primer that is specific to genetic loci of interest is used to specifically extract the DNA from these loci. Embodiments of the method include then constructing a secondary bisulfite sequencing library. In some embodiments, the proximity of the loci and the allele-specificity of the are validated using deeply sequenced targeted libraries.

In some embodiments, the methods are validated using publicly available SMAC-seq and Hi-C data collected from samples obtained from the sample cell line, cell sample, or tissue specimen. In some embodiments, the data are used to validate the genetic loci of interest. That is, in some embodiments, the loci without SNP overlapped nearby (non-SNP anchors) discovered in GTAGMe-seq is also measured using one or more techniques such as SMAC-seq in order to determine whether allele-specific accessibility/methylation is identified using both methods. In some embodiments, the high-resolution Hi-C results obtained from the same cell line, cell sample or tissue sample are further analyzed in order to compare contact frequency between the linked SNP-anchors and non-SNP anchors measured by GTAGMe-seq.

### EXAMPLES

### Example 1—GTAGMe-Sea: Joint Profiling of Genetic Variants, DNA Methylation, GpC Methyltransferase Footprints, and 3D Genome in the Same DNA Molecules

### Introduction

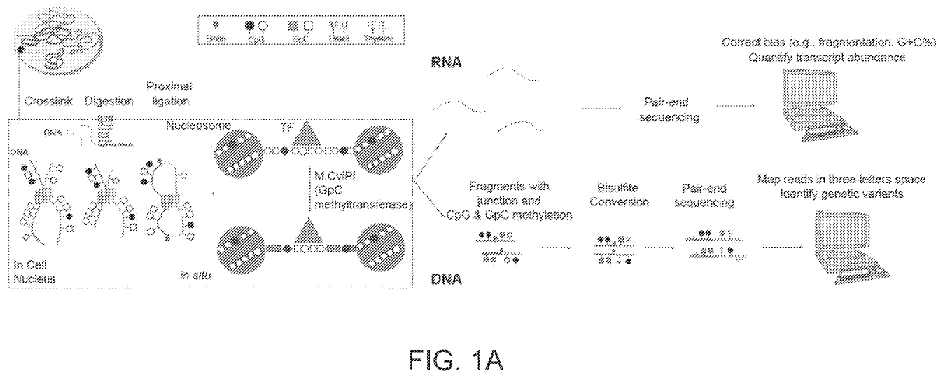

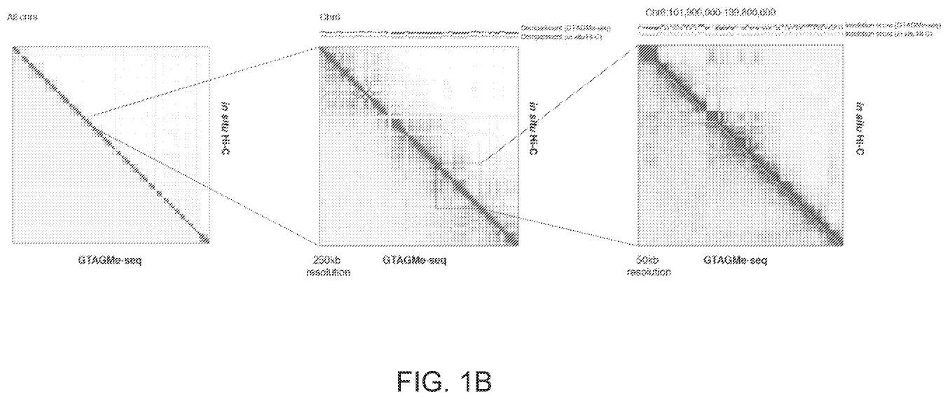

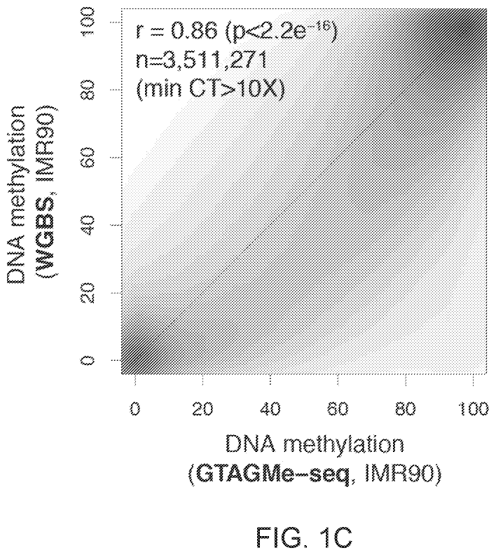

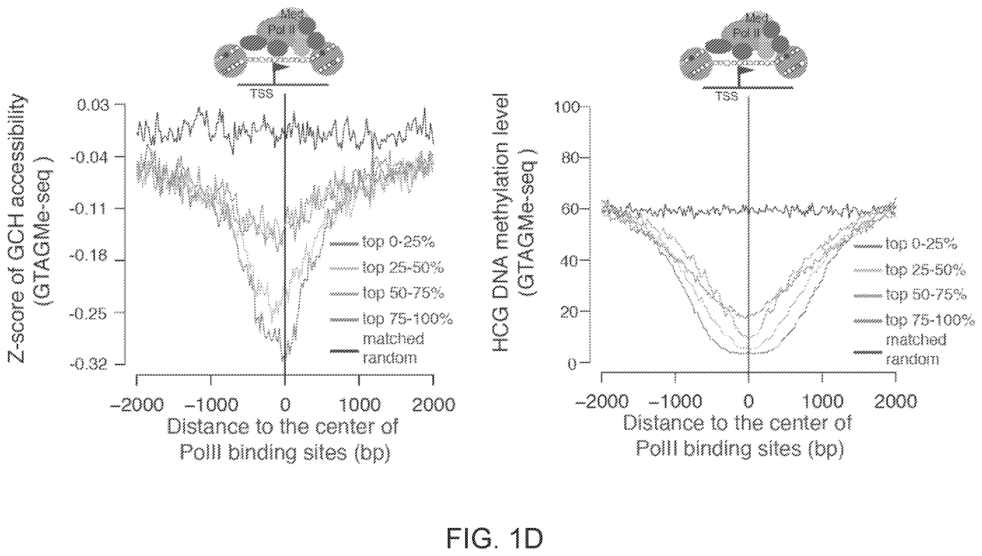

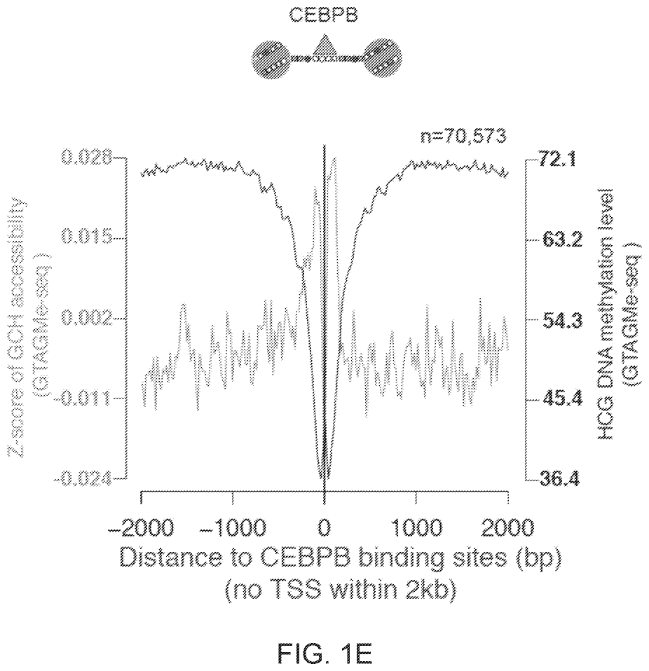

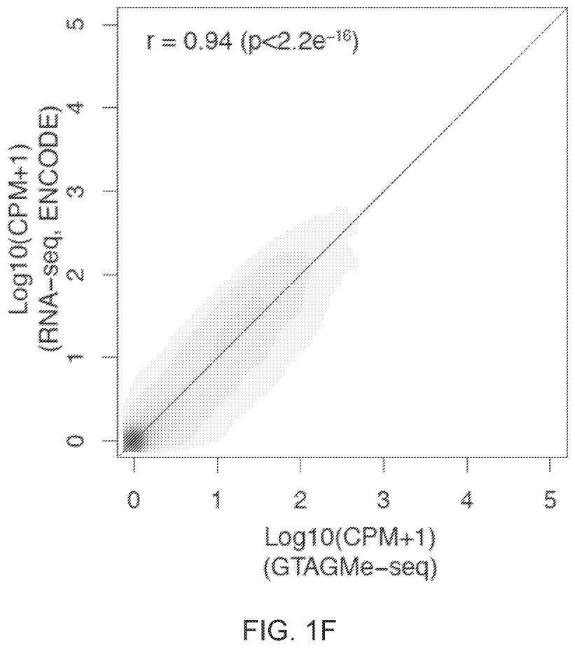

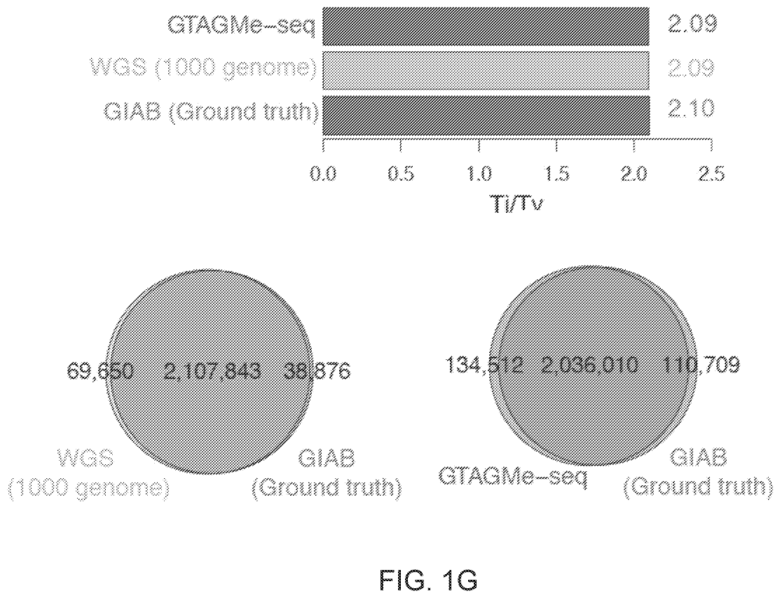

In Hi-C experiments, spatial proximity information is captured through restriction enzyme digestion and ligation of proximal genomic segments. After the ligation, nucleosomes and TFs are still crosslinked with DNA inside the nuclei, which can be footprinted by the exogenous GpC methyltransferase and detectable by the follow-up bisulfite sequencing, similar to the covalent genetic variants and endogenous CpG methylation. Therefore, based on the previously developed NOMe-seq and Methyl-HiC, the GTAGMe-seq (Genome, Transcriptome, GpC Accessibility, 3D Genome, and Methylome sequencing) technique of the present invention was developed in order to jointly profile multi-omics from the same DNA molecules, together with the transcriptome in the same assay (FIG. 1A).

### Methods

### Cell Culture

The human lung fibroblast cell line IMR-90 was purchased from ATCC and cultured in EMEM (ATCC) with 10% FBS (Gibco) and 1% penicillin/streptomycin. After reaching 70-80 confluence, cells were detached with 0.5% Trypsin and harvested by centrifuge at 300 g for 5 minutes. Human B lymphoblastoid cell line GM12878 was obtained from CORIELL INSTITUTE and cultured with RPMI-1640 supplemented with 15% FBS and 1% penicillin/streptomycin. GM12878 cells were harvested at a density around 0.7 M to 0.8 M/mL by centrifuge at 300 g for 5 minutes.

### GTAGMe-Seq

Cells were crosslinked with 1% formaldehyde for 10 minutes at room temperature, then quenched by adding 0.2 M Glycine. After washing with ice-cold PBS, cells were suspended in the nuclei isolation buffer with RNase inhibitors and incubated on ice for 1 hour. Subsequent to nuclei membrane permineralization, chromatin was digested using 100U DpnII (NEB, R0543L) overnight at 37° C. with RNase inhibitors. The next day, DpnII was inactivated, and nuclei suspension was cooled to room temperature and subjected to biotin fill-in. Proximal segments were in situ ligated using T4 DNA ligase (NEB, M0202) supplemented with RNase inhibitors. Then GpC methyltransferase footprint was performed by incubating the nuclei with M.CviPI (NEB, M0227L), and PBS was added to stop the reaction. After that, one-third of the nuclei were separated for RNA isolation using MagMAX^{TM} FFPE DNA/RNA Ultra Kit (Thermo A31881). The total RNA library was prepared using SMARTer@ Stranded Total RNA-Seq Kit v2 (TAKARA 634413). The rest of the nuclei were reverse crosslinked, DNA was precipitated and sonicated to average 400 bp followed by 1×AMPure beads cleanup. Biotin pulldown was performed using streptavidin beads, DNA on beads was end-repaired, d-A tailed, and ligated with truncated cytosine-methylated X-gene Universal Stubby Adapter (IDT). 0.1%-0.5% unmethylated lambda DNA was spiked-in. Bisulfite conversion was conducted using the EZ DNA Methylation-Gold Kit 223 (D5006). Bisulfite converted DNA library was amplified using truncated TruSeq™-Compatible Indexing Primer and KAPA HiFi Uracil+ ReadyMix (Roche, KK2801), supplemented with MgCl2. Library quality was determined by qPCR and BioAnalyzer 2000 (Agilent Technologies). Pooling of multiplexed sequencing samples, clustering, and sequencing was carried out as recommended by the manufacturer on Illumina HiSeq XTen or NovaSeq S6000 with the 150 paired-end. RNA-seq libraries or Phi-X (20%) were spiked in to overcome the imbalance of GC ratio of the bisulfite-converted DNA libraries.

### GTAGMe-Seq (DNA) Data Analysis

Raw reads were first trimmed by using Trim Galore! (v0.6.6, with Cutadapt v2.10)19 with parameters “--paired_end--clip_R1 5--clip_R2 5-three_prime_clip_R1 5--three_prime_clip_R2 5” to remove the adapters and low-quality reads. The clipped length was determined based on the composition of base pairs along the sequencing cycle by FastQC (v0.11.9). The reference genome (b37, human_glk_v37.fa) was in silico converted to make C/T reference (all Cs were converted to Ts) and G/A reference (all Gs were converted to As). Paired-end reads were mapped by our previously developed bhmem (v0.37)14 to each converted reference. Only uniquely mapped and mapping quality passed reads (mapQ>30) on both ends were joined. Reads marked as PCR duplicated reads were filtered. The reads with incompletely converted cytosine (WCH, W=A or T, H=A, C, or T) were filtered out as previously described. Joint read pairs with more than 20 kb insertion size were considered as long-range interactions and subjected to the following interaction analysis. Bam files were further converted to .hic files for the following 3D genome analysis similar to Hi-C data (details in the analysis of in situ Hi-C data). HCG methylation and GCH accessibility level were calculated by bissnp_easy_usage.pl with Bis-SNP (v0.90) in NOMe247 seq mode. Only bases with a quality score of more than 5 were included in the downstream methylation analysis. More details were implemented in Bhmem.java 248 with parameters “-outputMateDiffChr-buffer 100000”.

### GTAGMe-Seq (RNA) Data Analysis

Raw reads were first trimmed as paired-end reads using Trim Galore! (v0.6.6, with Cutadapt 253 v2.10) with parameters “--paired_end--clip_R1 15-clip_R2 15-three_prime_clip_R1 5--three_prime_clip_R2 5” to remove the adapters and low-quality reads. The clipped length was determined based on the composition of base pairs along the sequencing cycle by FastQC (v0.11.9). Both alignment-based (STAR, v2.7.6a20, and RNA-SeQC, v2.3.521) or alignment-free (Salmon, v1.5.122) approaches were tried. Salmon was finally used for the bias correction, visualization, and data analysis due to the slightly better concordance with the RNA-seq in ENCODE. The reference transcriptome was obtained from Gencode (v33lift37) and indexed by Salmon with k=31. The parameter used for Salmon quantification was: “quant--seqBias--gcBias--posBias-1 A--validateMappings”. The differential expression analysis was performed on gene level by the edgeR (3.32.1) package in R (4.0.5). Only genes with FDR<0.05 and log 2 fold change >1 were considered as the differential expressed genes between IMR-90 and GM12878. The same RNA-seq analysis pipeline was applied to the total RNA-seq from ENCODE.

Analysis of WGBS data DNA methylation level in WGBS was directly downloaded from ENCODE and extracted from the sites that only overlapped with HCGs in the reference genome.

### Analysis of In Situ Hi-C Data

Arrowhead in Juicer tools (v1.22.01) is used to call TAD domains, with parameters “-r 10000-k KR-m 2000”. The result is a BEDPE file containing all identified domains. FAN-C(v0.9.21) is used to calculate insulation scores, with subcommand insulation and arameters” @10 kb@KR-w 500000”. To calculate A/B compartment scores, the eigenvector command was used in Juicer tools (v1.22.01) to obtain the first eigenvector of the Hi-C observed/expected correlation matrix, with parameters KR and BP 500000. Then the average GC contents was calculated for each 500 kb bin. With the GC contents and the eigenvector aligned, the sign of the eigenvector was flipped if needed so that high GC content regions are associated with positive compartment scores (compartment A), and low GC content regions are associated with negative compartment scores (compartment B).

Accurate Identfication of SNPs from GTAGMe-Seq

BisulfiteGenotyper in Bis-SNP (v0.90)11 was utilized to identify SNPs with a genotype quality score of more than 20 at GTAGMe-seq data (raw genotype). Further, VCFpostprocess was utilized to filter out poor quality SNPs (at the regions with more than sequencing depth 250×, with strand bias more than −0.02, with sequencing depth more than 40× and fraction of reads with mapping quality 0 more than 0.1, genotype quality divided by sequencing depth less than 1, with 2 SNPs nearby within the +/−10 bp window). Finally, the filtered SNPs were utilized for imputation and phasing at by Minimac4 (v1.0.2) and Eagle (v2.4.1) together with 1000G phase3 v5 panel (EUR population). Since NA12878 was already profiled in 1000G phase3 project, to avoid the potential bias, NA12878 was excluded from the panel for the imputation and phasing. Only biallelic SNPs loci were utilized for the imputation and phasing. Other parameters during the SNP imputation and phasing followed the similar steps as that recommended by Michigan Imputation Server. After the imputation, only biallelic SNPs with minor allele frequency more than 1% and R\hat{ }2 more than 0.3 were kept for the following analysis. WGS genotyped results (NA12878) were downloaded from the 1000G project (phase3). Gold standard genotype results (NA12878, a.k.a HG001) were downloaded from the Genome in a Bottle (GIAB) website. Bcftools (v1.10.2) was utilized to summarize the Ti/Tv ratio. Concordance in GATK (v4.1.9.0) was utilized to evaluate the concordance among gold standard (GIAB), WGS (1000G), and GTAGMe-seq.

### Correlation Analysis of GpC Methyltransferase Footprint in GTAGMe-Seq

Only read pairs with GCH methylation levels at both ends were kept for the analysis. For the regions of interest, such as HiCCUPS loops anchor regions, Pearson Correlation Coefficient (PCC) was calculated directly from the GCH methylation level of each read of the read pair, not by the average GCH methylation level at each end of anchor regions. Only read pairs spanning at least 20 kb genomic distance were considered for the analysis. As a control, all the read pairs within the same genomic regions, no matter whether they have long-range interaction or not, were randomly shuffled 100 times and then subjected to PCC calculation. Fisher's (1925) z, implemented in “cocor.indep.groups” at R package “cocor”, was used to assess the significance between observed PCC from interacted read pairs and PCC of the random read pairs. The scripts for this analysis are provided in MethyCorAcrossHiccups.java with parameters “-methyPattemSearch GCH-methyPattemCytPos 2-bsConvPattemSearch WCH-bsConvPattemCytPos 2-useBadMate-useGeneralBedPe”.

### Analysis of Long-Range Allele-Specific GpC Methyltransferase Footprint in Gtagme-Seq

Only read pairs with one end overlapped with heterozygous SNPs, and GCH methylation levels at both ends were kept for the analysis. At each heterozygous SNP position with enough reads covered at both reference alleles and alternative alleles, the p-value was calculated by Fisher Exact test and followed with FDR correction (Benjamini-Hochberg). Only loci with FDR<0.05 were considered as significant allele-specific loci. For SNP anchor-Only or non-SNP anchor-Only groups, the methylation differences between alleles should have FDR>0.95 at the insignificant anchor. The scripts for this analysis is provided in LongRangeAsm.java with parameters “-minDist 1000-coverageRef 2-coverageAlt 2-methyPattemSearch GCH-methyPattemCytPos 2-bsConvPattemSearch WCH-bsConvPattemCytPos 2-useBadMate-minMapQ”.

### Crosslink, Nuclei Isolation, and DpII Digestion

Cells were resuspended in ice-cold PBS at a concentration of 1 M/mL and crosslinked with 1% formaldehyde (Thermo Scientific 28906) for 10 min with gentle rotation at room temperature, followed by adding 0.2M Glycine and gently rotating in room temperature for 5 min to stop crosslink. After washing, cells were suspended in Hi-C nuclei isolation buffer (10 mM Tris-HCl pH 8.0, 10 mM NaCl, 0.2% NP-40, 1× cOmplete protease inhibitor (Roche 11873580001)) supplemented with 1 mM DTT, 0.02 U/μL SUPERase⋅In™ RNase Inhibitor (Thermo AM2694) and 0.4 U/μL RNaseOUT Recombinant Ribonuclease Inhibitor (Thermo Scientific 10777019) and incubate on ice for 1 hour. Nuclei were spun down at 2500 g for 5 minutes at 4° C. and washed once with the above nuclei isolation buffer and spun down at 2500 g for 5 minutes at 4° C. Then the nuclei membrane was permineralized with 50 μL 0.5% SDS at 62° C. for 10 minutes. Permineralization was stopped by adding 25μL 10% Triton-X100 and 145 μL H₂O. To digest the chromatin, 26.5 μL NEB buffer3.1, 100U DpnII (NEB R0543M), 0.08 U/μL SUPERase⋅In™ RNase Inhibitor, 0.16 U/μL RNaseOUT Recombinant Ribonuclease Inhibitor, and 1 mM DTT were added to the nuclei suspension and shaking overnight at 37° C.

### Biotin Fill-In and Proximal Ligation

DpnII was inactivated by incubation at 65° C. for 10 min and cool to room temperature for at least 10 min. Sticky ends were filled in with 0.05 mM Biotin-1,4-dATP, 0.05 mM dGTP, 0.05 mM dCTP and 0.05 mM dTTP by 0.13 U/μL Klenow (NEB M0210L) at 37° C. for 90 min with 500 rpm shaking, supplemented with 0.02 U/μL SUPERase⋅In™ RNase Inhibitor and 0.04 U/μL RNaseOUT Recombinant Ribonuclease Inhibitor and 1 mM DTT to prevent RNA degradation. Then proximal blunt-ends were ligated with 1.67 U/μL T4 DNA Ligase (NEB M0202L) supplemented with 1× T4 DNA Ligase buffer, 1% Triton-X100 and 0.1 mg/mL BSA (NEB B9000s) for 4 hr at room temperature with gentle shaking (300 rpm).

M.cviPI Treatment

After ligation, nuclei were pelleted at 2500 g for 5 min 50 at 4° C., followed by resuspension in 282 μL 1× GpC buffer. Then nuclei suspension was treated with 50 uL 4U/μL M.cviPI enzyme (NEB M0227L) supplemented with 1.5 μL 32 mM SAM, 150 μL 1 M sucrose and 17 μL 10×GpC buffer for 7.5 min at 37° C. To enhance the efficiency of GpC methyltransferase footprint profiling, nuclei suspension was again treated with 25 μL 4 U/μL M.cviPI supplemented with 1.5 μL 32 mM SAM for 7.5 minutes at 37° C. To stop the reaction, 5 μL ice-cold PBS supplemented with 0.02 U/μL SUPERase⋅In RNase Inhibitor and 0.04 U/μL RNaseOUT Recombinant Ribonuclease Inhibitor and 1 mM DTT were added. Then the nuclei suspension was split into two parts: 300 μL for RNA-seq library preparation, 1200 μL for whole-genome bisulfite library preparation.

### Nuclei RNA Isolation

Nuclei RNA was isolated with MagMAX FFPE DNA/RNA Ultra Kit (Thermo Scientific A31881) according to the protocol with minor modification. Briefly, 300 μL Protease Digestion Buffer and 10 μL Protease was added to 300 μL nuclei suspension, and incubated at 55° C. overnight and 90° C. one hour to reverse crosslink the RNA. Then the suspension was cooled down to room temperature for 15 minutes. Nuclei RNA was captured by adding 20 μL Dynabeads™ MyOne™ Silane (Thermo Scientific, 37002D) supplemented with 1000 μL binding solution and 1250 μL isopropanol, and shaking at 1000 rpm for 10 minutes at RT. The beads were collected by placing the sample-containing tube on a magnet for 5 minutes or until the supernatant was clear. Then the beads were washed once with a 500 μL RNA wash buffer and once with a 500 μL wash solution. DNA was digested by resuspending the Saline beads in 20 μL DNase, 10 μL DNase buffer, and 70 μL H₂O and incubating at RT for 20 minutes. To recapture the RNA, 200 μL binding buffer and 250 μL isopropanol were added and incubated at RT for 10 minutes with 1000 rpm shaking. The beads were again washed once with the RNA wash buffer and twice with the wash solution. After briefly air-drying the beads, nuclei RNA was eluted with 30 μL nuclease-free H₂O.

### RNA-Seq Library Preparation.

10 ng nuclei RNA was used for library preparation using SMARTer® Stranded Total RNA-Seq Kit v2-Pico Input Mammalian-96 Rxns (TAKARA 634413) kit.

### Reverse Crosslink, DNA Precipitation, and Sonication.

Nuclei DNA was first reverse crosslinked by adding 50 μL 20 mg/mL proteinase K and 120 μL 10% SDS, then incubating at 55° C. for 30 minutes. The transcription factor was disload by adding 130 μL of 5 M NaCl and then incubated at 68° C. for 4 hours. Tubes were cooled to room temperature, and nuclei DNA was precipitated at −20° C. overnight with 1.6× EtOH and 0.1× NaAc. The next morning, nuclei DNA was collected by spin down at max speed for 15 minutes at 4° C., and washed twice with fresh 80% EtOH, then dissolved in 130 μL 10 mM Tris-HCl pH 8.0. The DNA was sonicated to an average of 400 bp with Covaris M220 using the following parameters: peak power 30, duty factor 10, cycles/burst 200, duration 60 seconds. A 1× AMPure size selection was performed to get rid of the small fragments, and nuclei DNA was then suspended in 300 μL 10 mM Tris-HCl pH 8.0.

Biotin Pull-Down, End-Repair, dA-Tailing, and Library Preparation

Dynabeads My One T1 Streptavidin beads (Invitrogen 65602) was washed once with 1× tween wash buffer (5 mM Tris-HCl ph7.5, 0.5 mM EDTA, 1M NaCl, 0.05% Tween-20) and suspended in 300 μL 2× binding buffer (10 mM Tris-HCl pH 7.5, 1 mM EDTA and 2 M NaCl). Then biotin pull-down was performed by rotation at room temperature for 15 minutes. After that, beads-DNA was washed twice with a 1× tween wash buffer at 55° C. with mixing. Then end-repair was performed, and unligated fragments were removed with 0.5 mM dNTP (Thermo R1122), 0.5 U/μL T4 PNK (NEB M0201L), 0.12 U/μL T4 DNA polymerase (NEB M0203L), 0.05 U/μL Klenow (NEB M0210L) in 100μL 1× T4 DNA ligase buffer at room temperature for 30 minutes with 300 rpm mixing. DNA was washed twice at 55° C. with mixing. dA-tailing was performed 0.5 mM dATP, 0.25 U/μL Klenow (e 100 xo-) (NEB M0212L) in 100 μL 1×NEB buffer 2 at 37° C. for 30 min. DNA was washed twice at 55° C. with mixing. 0.9 μM truncated cytosine-methylated X-gene Universal Stubby Adapter (IDT) was added by Quick Ligase in 50 μL 1× Quick Ligase buffer at RT for 15 minutes. Nuclei DNA was washed twice with 1× tween wash buffer at 55° C. followed by washing twice with 100 μL 10 mM Tris-HCl pH 8.0. Finally, DNA binding streptavidin beads were resuspended in 20 μL 10 mM Tris-HCl pH 8.0.

### Bisulfite Conversion

Spike in 0.1% 400 bp stubby ligated unmethylated lambda DNA into each DNA sample before bisulfite conversion. Bisulfite conversion was performed with EZ DNA Methylation-Gold Kit (Zymo D5006). DNA was separated from streptavidin beads after CT conversion.

### Library Amplification and Sequencing

The Bisulfite converted library was amplified using 100 ng input with KAPA HiFi Uracil+ ReadyMix (KAPA KK2801), 0.5 μM truncated TruSeq™-Compatible Indexing Primer, 2.5 mM MgCl₂. The following cycles were performed: 98° C. 45 seconds; 98° C. 15 seconds, 60° C. 30 seconds, 72° C. 30 seconds (10-15 cycles); 72° C., 1 minutes. After pooling and quality control, sequencing was performed at HiSeq-XTen or NovaSeq S6000 platform with 150 paired ends.

### Results

Briefly, after the crosslinking, we isolated nuclei and treated them with a methylation insensitive restriction enzyme (e.g., DpnII) to digest DNA. Proximity ligation was used to capture the long range interactions in the 3D genome topology. GpC methyltransferase (M.CviPI) was further utilized to footprint the chromatin accessibility. After reversing crosslinks, RNA molecules were extracted for total RNA-seq. The DNA molecules were isolated with the follow-up bisulfite conversion and paired-end sequencing to obtain covalent endogenous cytosine methylation and exogenous GpC methyltransferase footprint information. Finally, the previously developed computational method, Bis-SNP, was improved to accurately identify the genome-wide single nucleotide polymorphisms (SNPs) from the bisulfite converted reads.

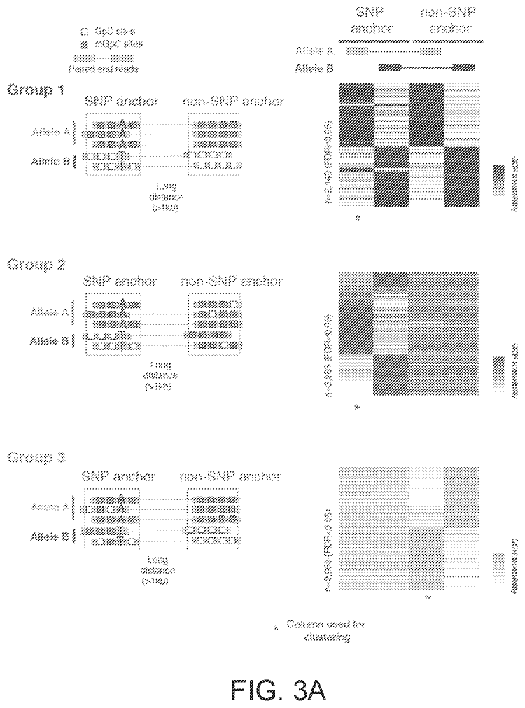

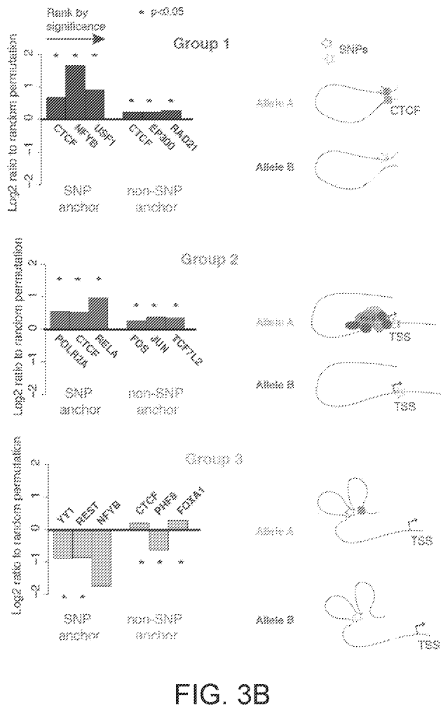

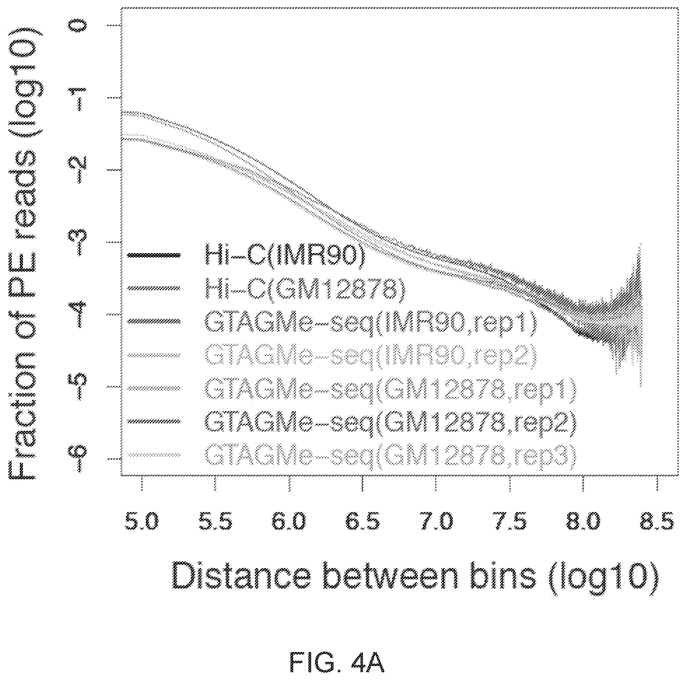

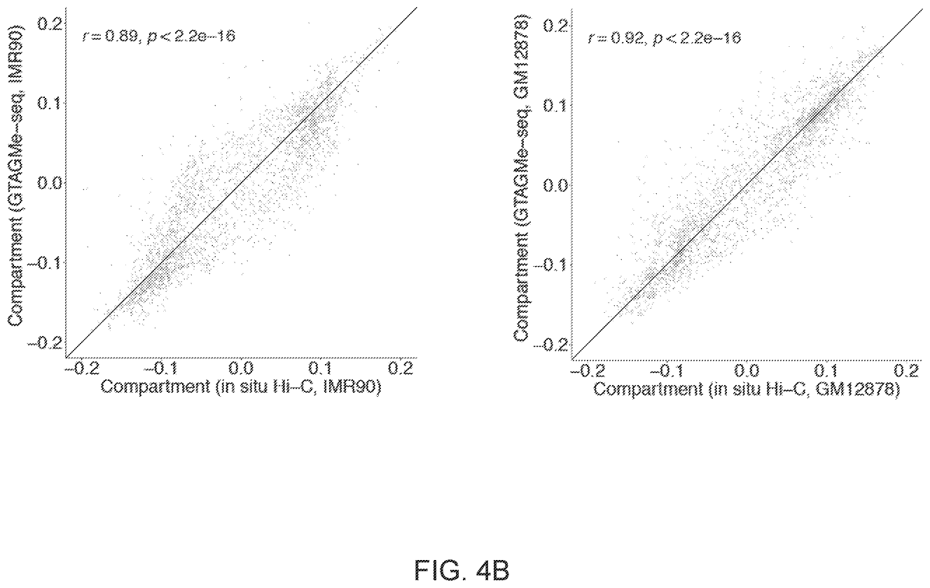

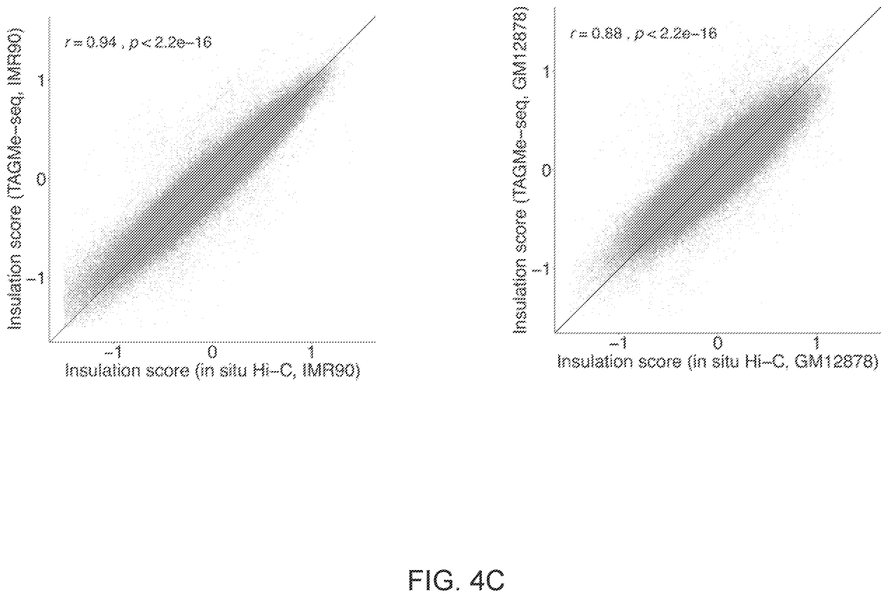

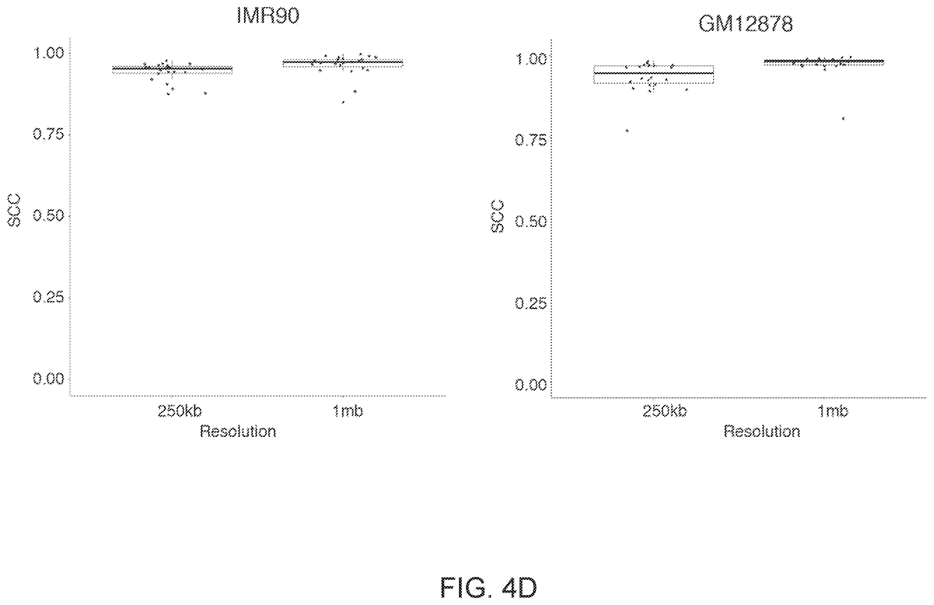

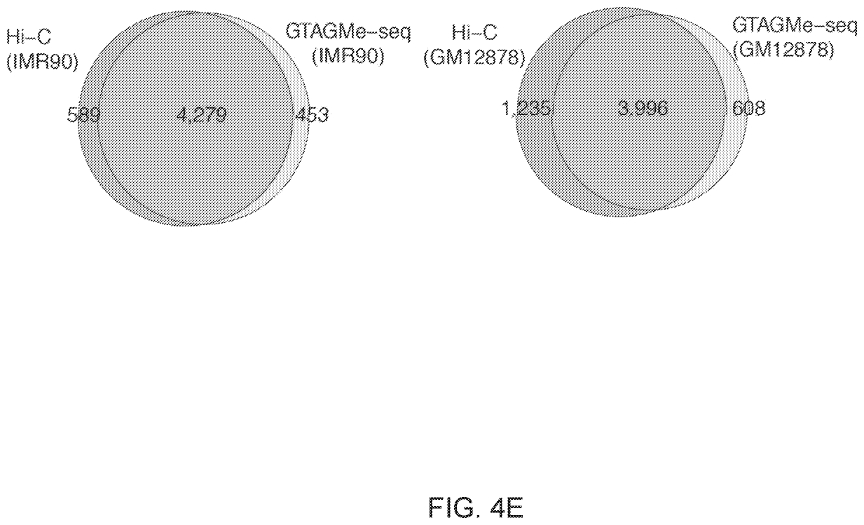

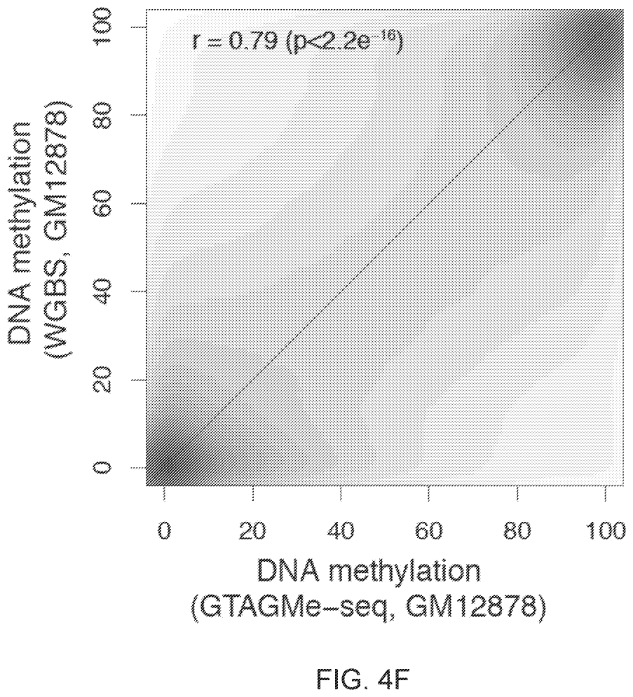

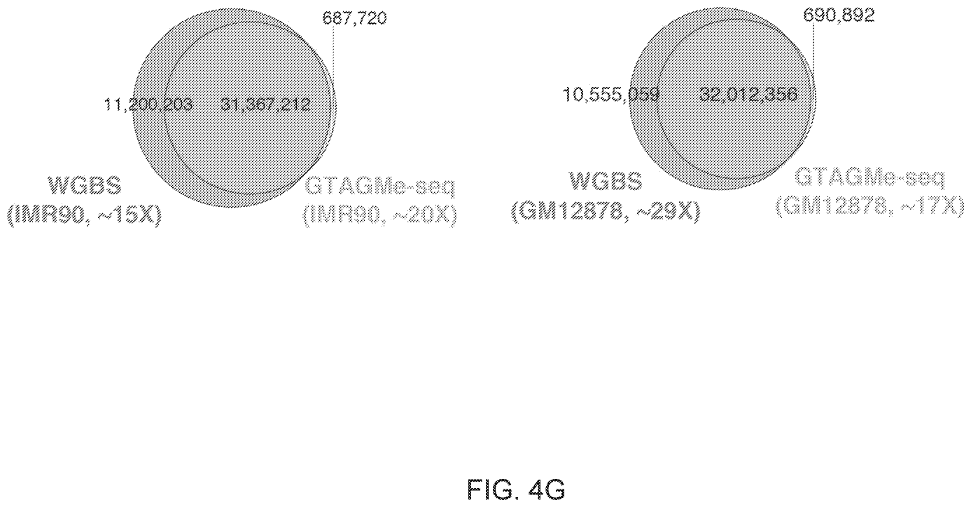

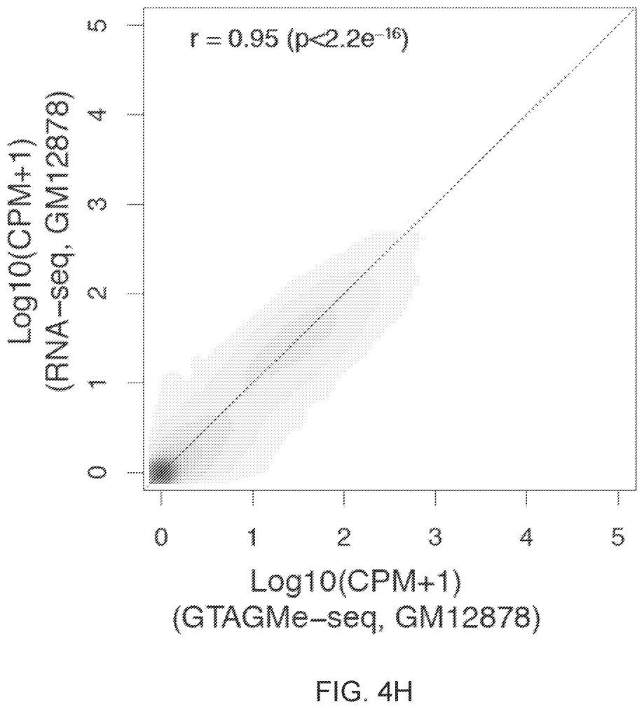

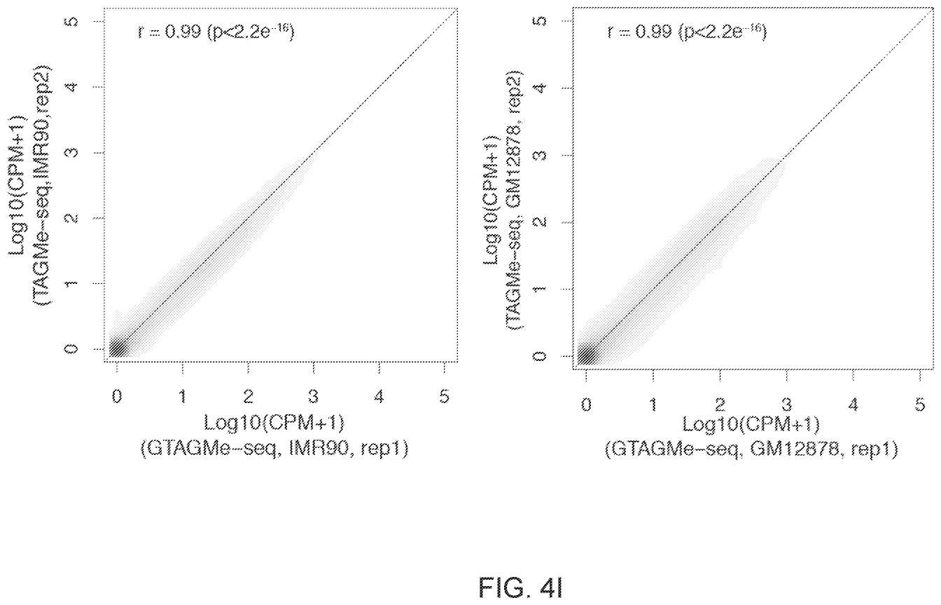

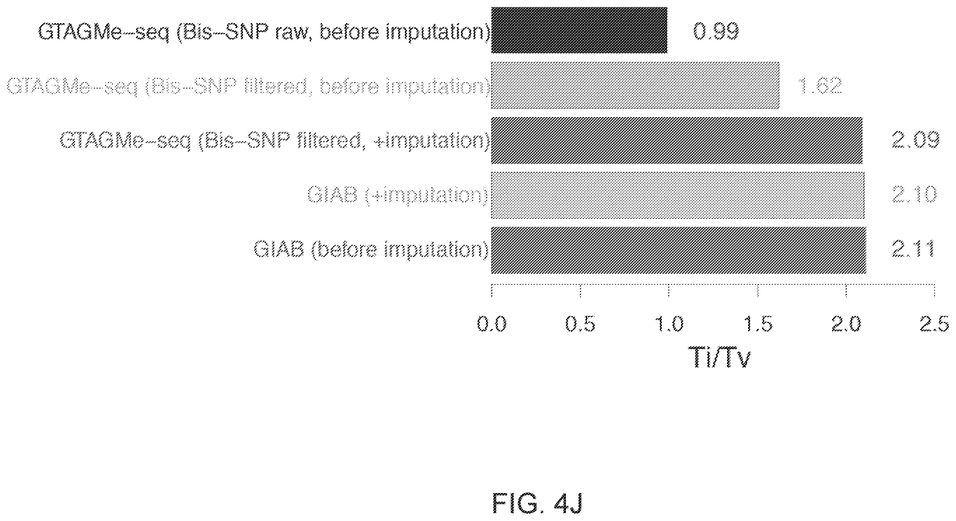

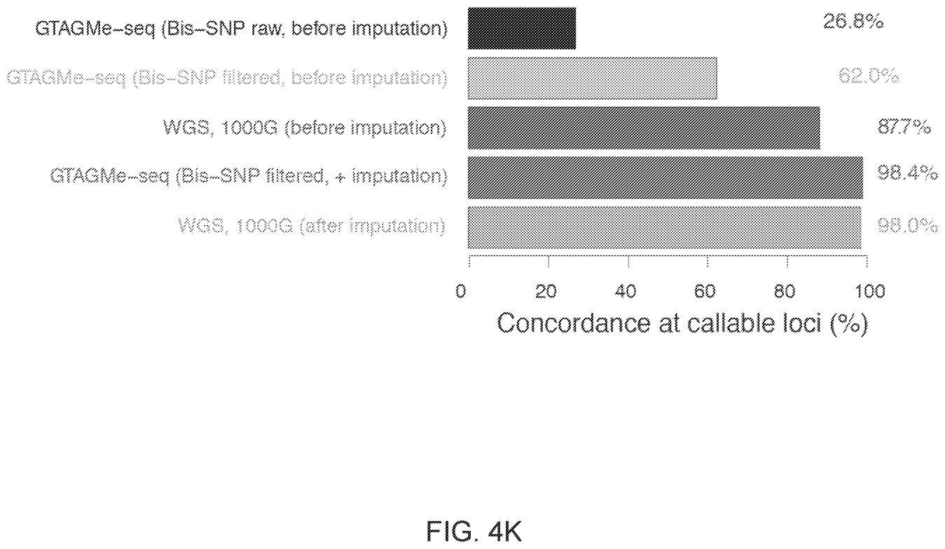

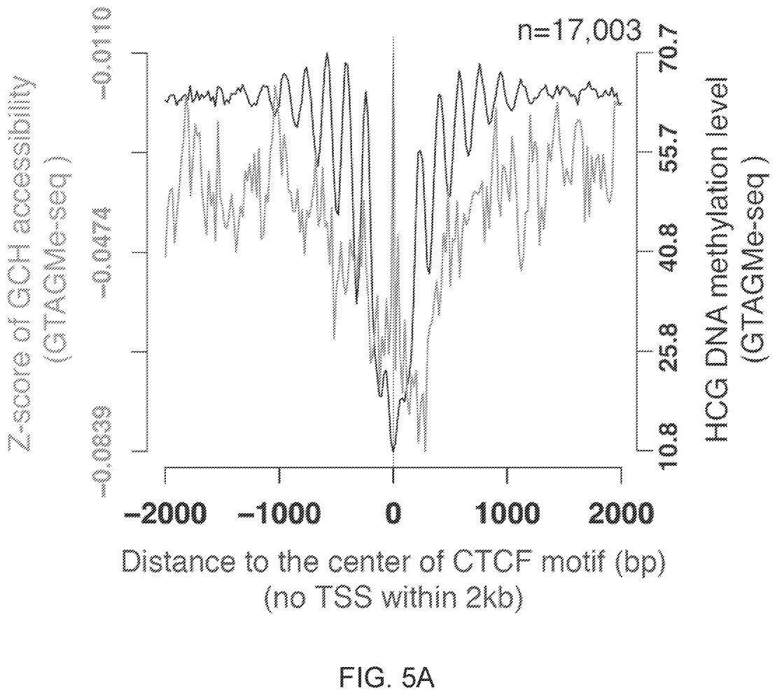

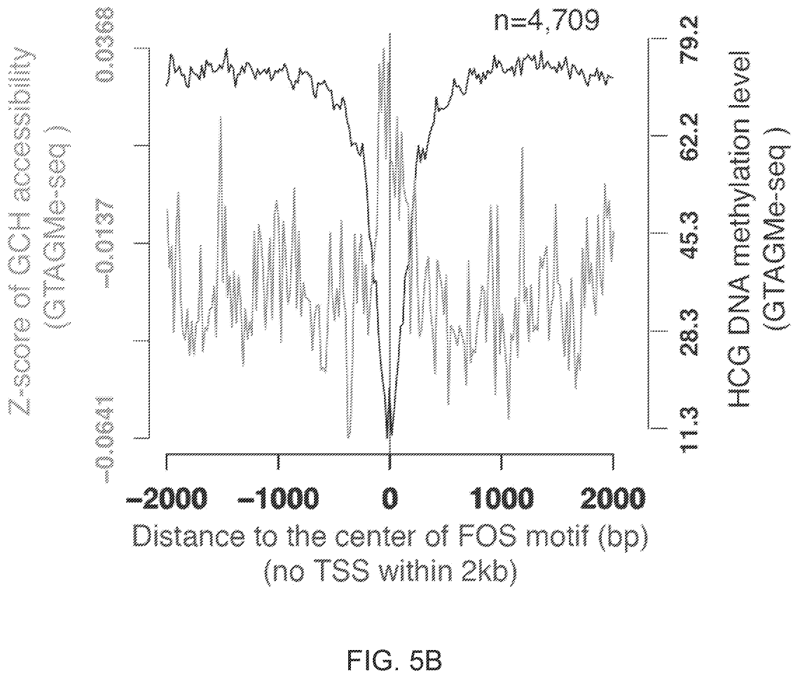

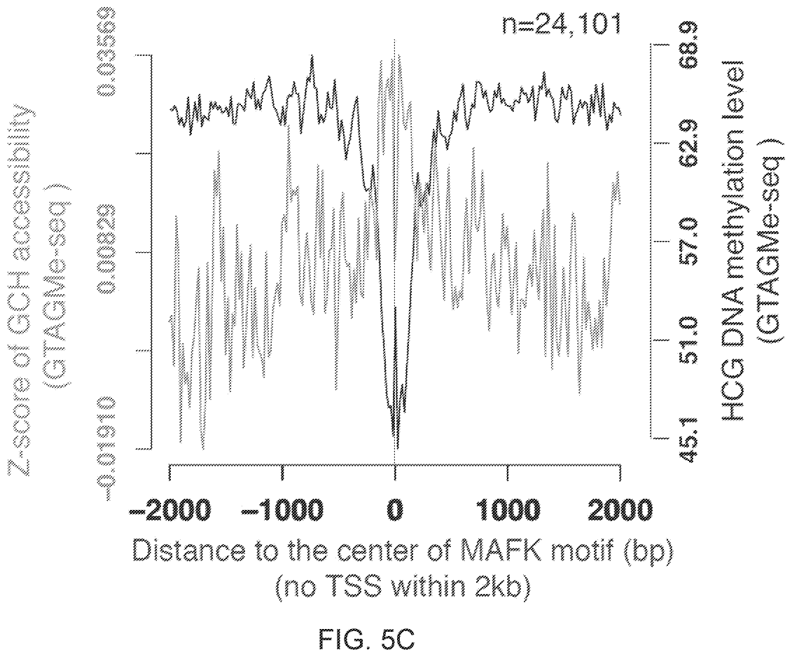

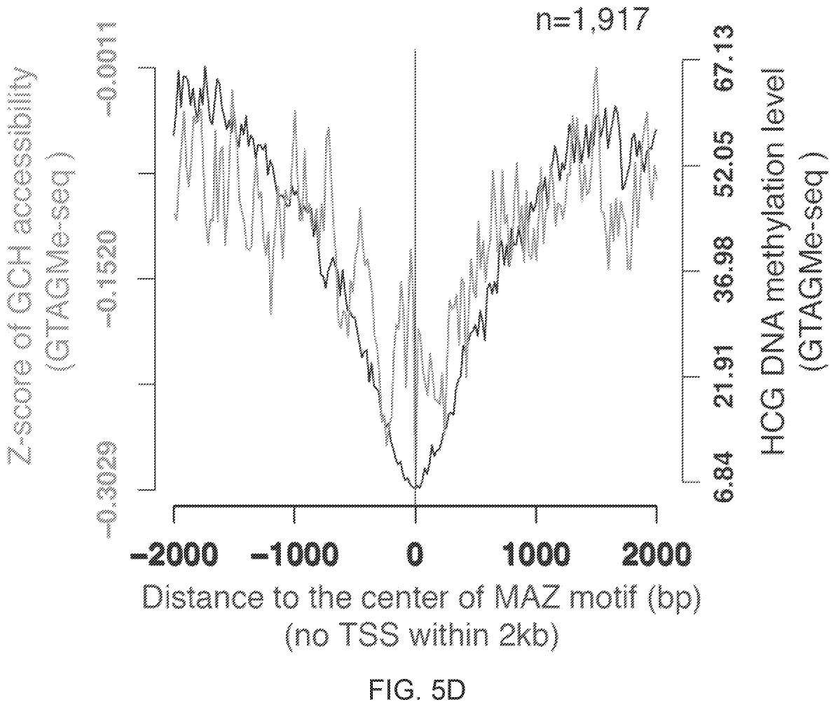

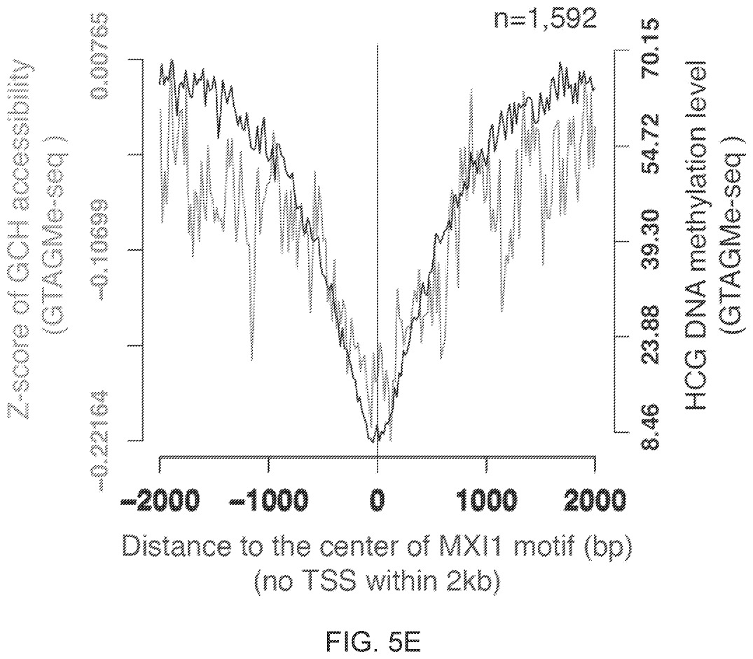

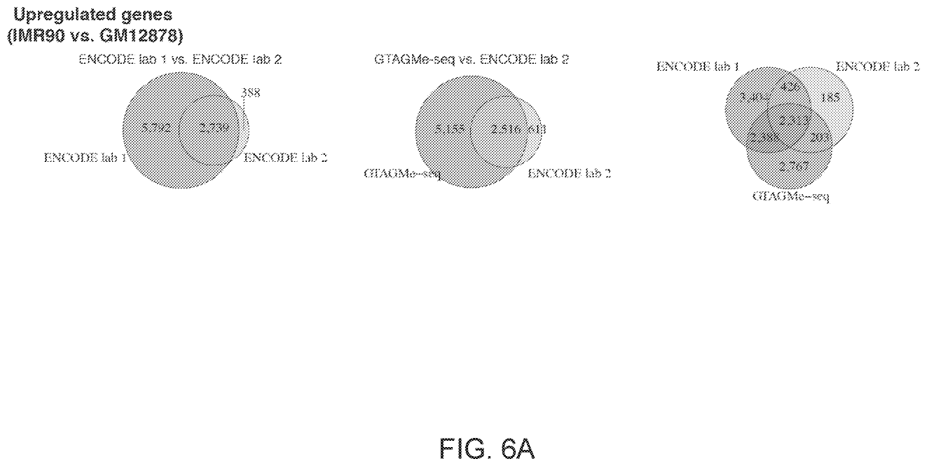

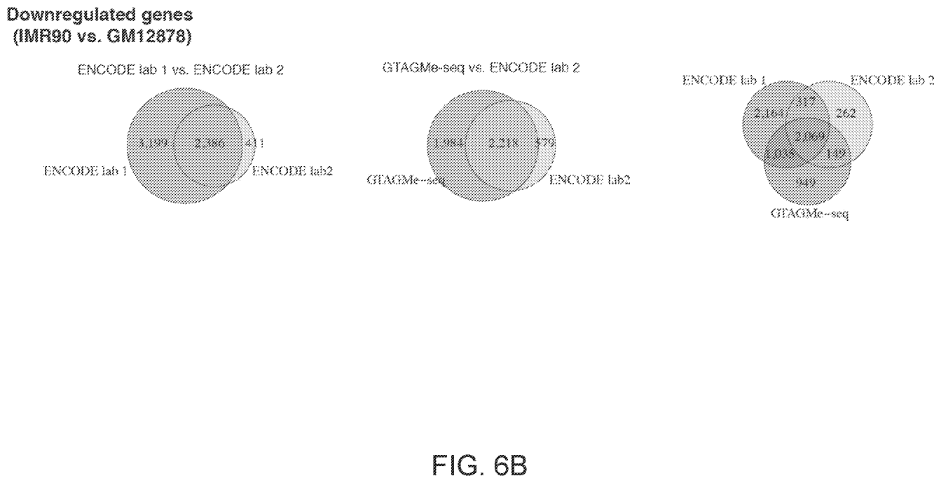

To demonstrate the performance of GTAGMe-seq, it was applied to the human lung fibroblast cell line (IMR-90) and B-lymphoblastoid cell line (GM12878)), which have both been comprehensively profiled publically. First, the measurement of the 3D genome was compared with in situ Hi-C results in the same cell lines. The contact matrix is highly similar to that from in situ Hi-C (FIG. 1B, FIG. 4D), with the indistinguishable distribution of contact probabilities (FIG. 4A). Across different resolutions, comparable compartment score, insulation score, and topologically associated domains (TADs) was identified from the two datasets (FIGS. 4B-C, E). Next, the measurement of DNA methylation was compared with WGBS from the same cell lines. To avoid the ambiguity of endogenous CpG methylation and exogenous GpC methyltransferase footprint at GpCpG (GCG) sites, GCG sites were excluded for the follow-up measurement of CpG methylation and GpC methyltransferase footprint as was done in the NOMe-seq paper. GTAGMe-seq captured 31-32 million HCGs (H=A, C, or T) in the human genomes and the methylation level of these HCGs was highly consistent with that from WGBS (FIG. 1C, FIG. 4F-G). Similar to Methyl-HiC, GTAGMe-seq captured ˜25% fewer HCGs compared to WGBS. Third, the measurement of GpC methyltransferase footprint (GCH accessibility) we tested at the known TFs binding sites. Notably, nucleosomes and TFs are both crosslinked with DNA. Part of the open chromatin regions will still show the low GCH accessibility level when TFs are bound, which is different from the conventional NOMe-seq without the fixations. A concordant lower level of GpC methyltransferase footprints was observed at PolII binding sites with a higher TF occupancy level measured by PolII ChIP-seq in the same cell line (FIG. 1D). The expected GCH footprint patterns and HCG methylation level was also observed at TF binding sites that usually bind distantly to the transcriptional starting sites (TSS) FIG. 1E, FIG. 5). Moreover, the measurement of the transcriptome was compared with total RNA-seq from the same cell lines. Messenger RNA (mRNA) is highly fragmented after the formaldehyde fixation step (crosslink). With the computational correction of the possible bias at fragmentation and G+C %, the transcriptome is generally concordant with that from total mRNA seq, in terms of absolute quantification (FIG. 1F, FIG. 4H-4I) and differentially expressed genes (FIG. 6). Finally, the measurement of SNPs (GM12878) was compared with deep WGS from the 1000 genome project on the same individual (NA12878, 127 a.k.a. HG001). The performance was evaluated by the ground truth genotype results from the same individual at the Genome in a Bottle Consortium (GIAB), which are well characterized by the integration of multiple technologies. Results with improved Bis-SNP at GTAGMe-seq showed similar high performance as that from WGS (FIG. 1G, FIG. 4J) and very high accuracy at sites with genotype callable (FIG. 4K). These results, taken together, demonstrated that GTAGMe-seq can simultaneously and accurately profile 3D genome, DNA methylation, GpC methyltransferase footprint, transcriptome, and SNPs in the biological samples. Next the inventors asked if the genomic regions linearly separated but positioned proximity in 3D space would have coordinated footprint status. The GCH (H=A, C, or T) methyltransferase footprint was analyzed on GTAGMe-seq reads separated in chromatin loop anchors (FIG. 2A). Indeed, the GCH methyltransferase footprint in GTAGMe-seq read pairs mapping to separated loop anchors showed a similarly high correlation as that mapping to the local regions (<1kb, FIG. 2B). To ensure the concordance is not due to the similar level of GCH methyltransferase footprint at the loop anchors, the links of read pairs were shuffled at the loop anchors while maintaining the average GCH methyltransferase footprint at loop anchors. The shuffled read pairs indeed showed significantly much less correlation (FIG. 2A-2B, Fisher's (1925) z test, p<2.2e-¹⁶). To further demonstrate the utility of GTAGMe-seq, it was utilized to analyze the long-range allele specific GpC methyltransferase footprint. GTAGMe-seq reads were separated into two groups by their parent-of-origin at the heterozygous SNPs (SNP anchors, FIG. 3A). Since the sequencing reads come from the same single DNA molecules, the other ends of the read pairs were also separated into two alleles, even though they are not overlapped with the SNPs and mapped over large genomic distances (non-SNP anchors, FIG. 3A). Thus, the long-range allele-specific GCH methyltransferase footprint was able to be analyzed. There were 149 three groups of allele-specific footprint loci identified based on their differences of GCH accessibility between the two alleles at both anchors (Group 1, n=2,149, FDR<0.05 at both anchors), or only at SNP anchors (Group 2, n=3,285, FDR<0.05), or only at non-SNP anchors (Group 3, n=2,963, FDR<0.05). Further, to understand the function of these long-rang allele-specific footprints, the enrichment of the anchors over different known TF binding sites was checked. Group 1 showed the highest enrichment of CTCF binding sites at both anchors (FIG. 3B, FIG. 7), suggesting the disruption of the CTCF binding by genetic variants at the SNP anchor may also disrupt the loop structure and thus the binding activities at the non-SNP anchor in spatial proximity. Group 2 exhibited the highest enrichment of PolII binding sites at the SNP anchors, but enrichment of different TFs at the non-SNP anchors, which usually bind to the distal enhancer regions. Group 3 showed the highest depletion of TF binding sites that are usually bound to the promoter regions. Genome-wide association studies (GWAS) have associated common complex diseases with hundreds of thousands of genetic variants. Due to the linkage disequilibrium (LD), many SNPs near the tag SNPs in the GWAS studies are also identified to be significantly associated with the traits. The local CRE activities, especially the imbalanced CREs activities linked with the switching of risk and non-risk alleles nearby, are often utilized to prioritize the potential causal SNPs within the LD blocks. The Group 3 long-range allele-specific footprint may suggest that the switching of risk and non-risk allele does not need to affect the local chromatin accessibility but instead the activities of distant CREs in spatial proximity. Indeed, it was that observed the overlap of SNP anchors at Group 3 in GM12878 (B-lymphoblastoid cell) with three significant GWAS loci (rs1885276 with 4.157e-⁰⁹, rs6431262 with 2.023e-¹², and rs3805683 with 2.267e-²³) that are associated with inflammatory bowel diseases (IBD). IBD is found to be driven by the malfunctions of the B cells, but the annotation of chromatin states near these three regions are all heterochromatin regions in GM12878 cells. Thus, instead of methylation or accessibility QTLs mapping that required multiple samples, GTAGMe-seq here provides an alternative approach to link SNPs with the variations of distal CREs in spatial proximity at a single assay. Overall, a method to simultaneously study the genetic variants, DNA methylation, GpC methyltransferase footprint, and 3D genome in the same DNA molecules together with the transcriptome in the same assay has been developed. Combining multiple assays allowed one to uncover the coordinated GpC methyltransferase footprint and long-range allele-specific footprint at genomic regions that are far away in linear DNA sequences but spatially close in the nucleus.

### Example 2—Sinale-Nuclei GTAGMe-Sea to Jointly Profile Multi-Omics in the Same Nuclei

Based on the bulk and single-cell results, the single molecule GTAGMe-seq method was extended to single-nuclei GTAGMe-seq (snGTAGMe-seq) as described herein.

### Methods

The preliminary GTAGMe-seq protocol was extended to the single-nuclei level at IMR-90 cell lines.

An initial sample was processed with ˜5 million cells as described in the methods of Example 1. As described above, the restriction enzyme treatment, proximal ligation, and GpC methyltransferase footprint (MCviPI), were applied here. However, at the biotin labeling step, dATP was used instead of biotin-14-dATP, similar to scMethyl-HiC. After the GpC methyltransferase treatment, flow cytometry was utilized to sort the single nuclei into 96 or 384 wells. After that, the crosslink was reversed, and single nucleus mRNA was separated out by the oligo-dT linked magnetic beads in each well. The supernatant containing nucleus genomic DNA (gDNA) was transferred to a new nuclease-free 96 or 384-well plate. Since the widely used Tn5 transposases-based Nextera kit is not compatible with the highly fragmented RNA for library preparation, utilize the other enzymatic fragmentation and adapter adding strategy (NEB Kit) was utilized with customized modifications to synthesize cDNA, the adapter was added, and the libraries were prepared. The gDNA in each well was processed similar to how was done in scMethyl-HiC. Basically, 0.1% fragmented lambda DNA was spiked in to assess the bisulfite conversion rate in each sample, and bisulfite conversion was carried out using EZ Methylation Direct Column kit. The 4-multiplexed P5 adapter was added by random priming using Klenow exo-(50 U/ul). The Exonuclease I and Shrimp Alkaline Phosphatase was used to digest unused random primer and inactivate dNTPs, following a 1× ratio AMPure beads purification. Further, ACCEL-NGS ADAPTASE MODULE (Swift 33096) was used to add the P7 adapters. The DNA libraries was amplified using corresponding indexed primers. Both DNA and RNA Libraries were first quantified and QC by qPCR, Qubit, and BioAnalyzer 2000. Further, some randomly selected libraries were sequenced with shallow coverage (˜2 million reads) at the Illumina MiSeq platform. Since bisulfite conversion imbalance the G+C % ratio in the library, DNA libraries were pooled together with 20% PhiX or RNA libraries and sequenced deeper with 150 bp paired-end sequencing at the Illumina HiSeq 4000 or NovaSeq 6000 S4 platform

### Results

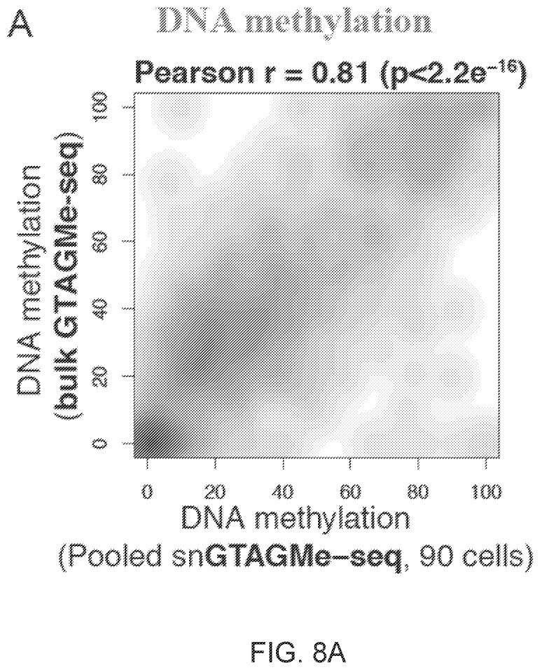

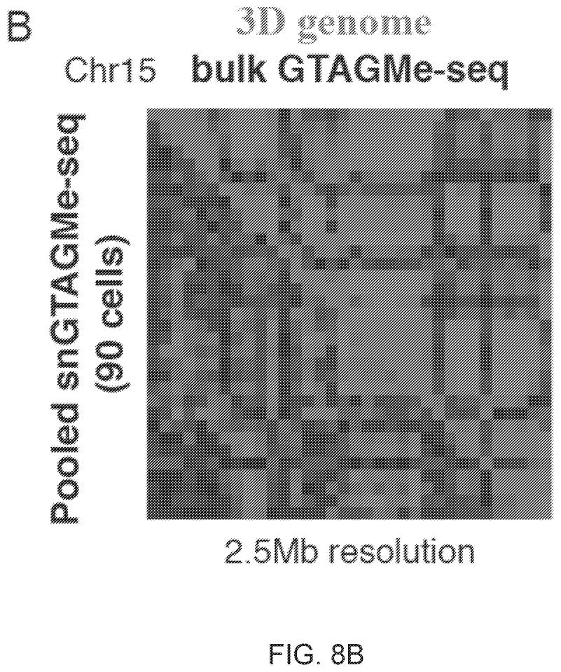

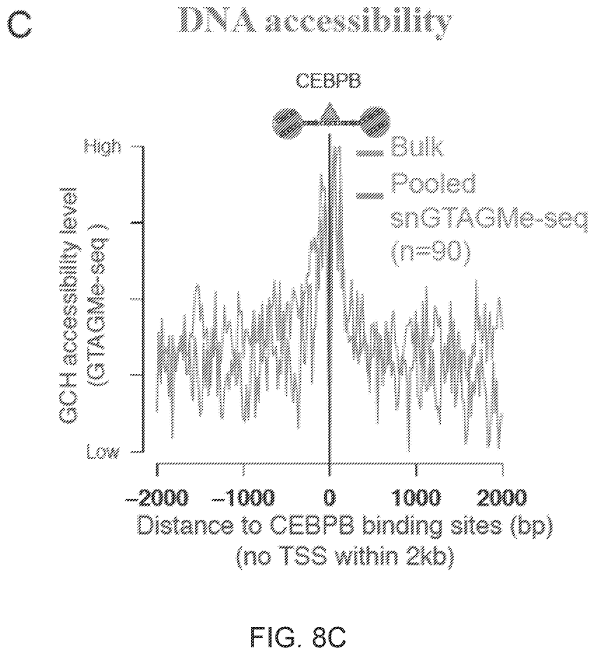

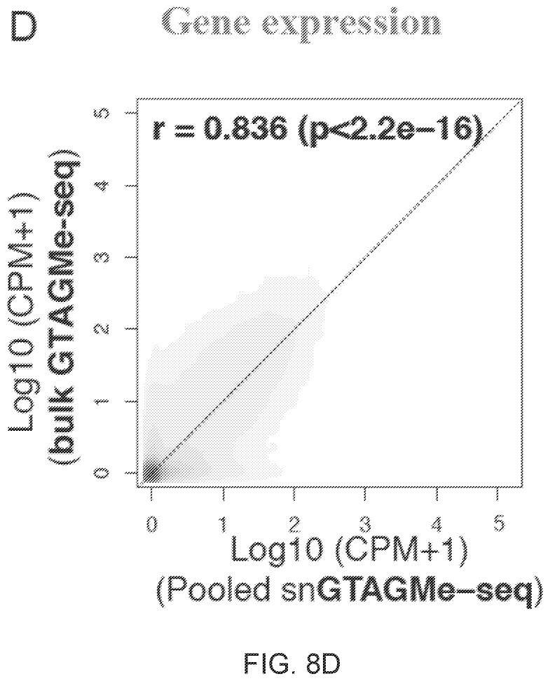

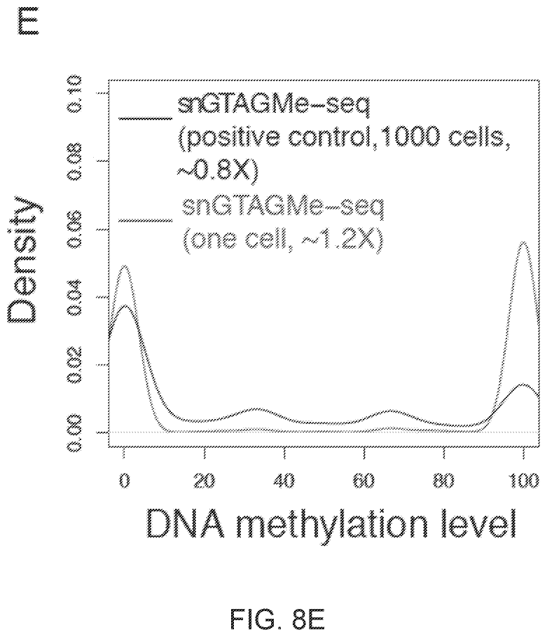

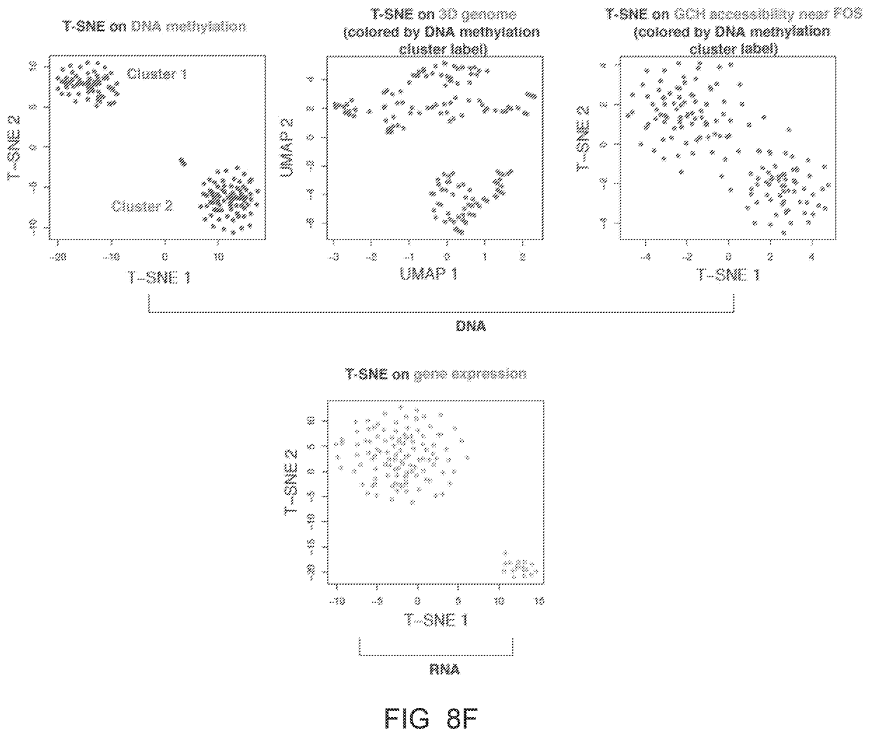

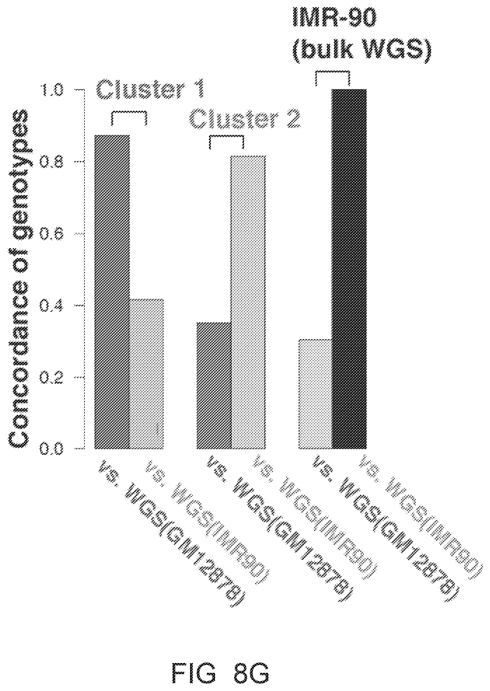

The pooled snGTAGMe-seq (n=90 cells, pooled computationally) showed high concordance across different molecular measurements with bulk GTAGMe-seq data at the IMR-90 (FIG. 8A-D). The distribution of DNA methylation levels in single-cell libraries (FIG. 4E) showed mostly binary methylation level per base (DNAm=0% or 100%) compared with the 1000-cells library (positive control) in the same batch at a similar coverage. This result indicated that we indeed captured the epigenetics at the single-cell level rather than the multi-cells level. The equal number of GM12878 and IMR-90 nuclei were further randomly mixed and then the snGTAGMe-seq was performed (n=151 cells). The embedding results at each molecular measurement showed distinguished two clusters (FIG. 8F). The cells were further pooled within each cluster and compared their genotyping results with that from WGS in two cell lines. The results suggested that the cellular heterogeneity was indeed recovered in this mixed cell population (FIG. 8G).

1. A system for detecting long-range cis-regulatory element (CRE) activities in a nucleic acid molecule obtained from a cell sample, the system comprising:
one or more components for measuring multi-omics from a single nucleic acid molecule obtained from the cell sample, wherein the multi-omics measurements comprise one or more of: three-dimensional chromosomal conformation, CpG methylation, GpC accessibility, single nucleotide polymorphisms (SNPs); or one or more combinations thereof, and
one or more components for measuring a transcriptome of the cell sample;
wherein the system is capable of profiling a plurality of long-range CREs within the nucleic acid molecule obtained from the cell sample.

2. The system of claim 1, wherein the cell sample comprises a bulk cell sample.

3. The system of claim 1, wherein the cell sample comprises a single cell sample.

4. The system of claim 1, wherein the one or more components for measuring three-dimensional chromosome conformation perform in situ methyl-HiC.

5. The system of claim 1, wherein the one or more components for measuring CpG methylation perform one or more of bisulfite conversion and paired-end sequencing.

6. The system of claim 1, wherein the one or more components for measuring the GpC accessibility comprises perform GpC methyltransferase foot-printing.

7. The system of claim 6, wherein the GpC methyltransferase foot-printing is performed using one or more GpC methyltransferases comprising M CviPI.

8. The system of claim 1, wherein the one or more components for measuring SNPs perform Bis-SNP analysis, a PairHMAM based analysis, or a combination thereof.

9. The system of claim 1, wherein the one or more components for measuring transcriptome perform an allele-specific transcriptome comparison of the two alleles of a single chromosome.

10. A method for detecting long-range CREs, the method comprising:
obtaining a cell sample;
performing nucleic acid cross-linking using one or more techniques;
isolating a nuclear sample from the cell sample;
measuring the methyltransferase footprint of the nuclear sample,
separating the nuclear samples into a first aliquot and a second aliquot,
preparing one or more RNA libraries from the nuclear sample in the first aliquot; and,
preparing one or more DNA libraries from the nuclear sample in the second aliquot.

11. The method of claim 10, wherein the cell sample comprises a single cell sample.

12. The method of claim 10, wherein the cell sample comprises a bulk cell sample.

13. The method of claim 12, wherein the DNA library is a bisulfite converted DNA library.

14. The method of claim 13, further comprising paired-end sequencing the bisulfite converted DNA library.

15. The method of claim 12, further comprising quantifying the transcript abundance in the first aliquot.

16. The method of claim 15, wherein the transcript abundance is determined using one or more of alignment-based techniques, alignment-free techniques, or both alignment-based and alignment free techniques.

17. The method of claim 12, wherein the RNA library is prepared using one or more Tn5-transposase-based techniques.

18. The method of claim 11, wherein the RNA library is prepared using one or more one or more combinations of enzymatic fragmentation and adaptor addition.

19. The method of claim 11, wherein the one or more DNA libraries are deeper sequences using one or more techniques comprising 150 bp paired-end sequencing.

20. A computation method for analyzing a single-molecule nucleic acid sample comprising processing a cell sample using the system of claim 1 for characterizing or quantifying one or more of DNA methylation, GCH methyltransferase accessibility, three-dimensional genomic conformation, transcriptome, and one or more combinations thereof.
